(19) United States
(12) Patent Application Publication
(10) Pub. No.: US 2025/0218532 A1
(43) Pub. Date: Jul. 3, 2025

(54) SYSTEMS AND METHODS FOR CANCER THERAPY MONITORING

(71) Applicant: Predicine, Inc., Hayward, CA (US)

(72) Inventors: Pan DU, Dublin, CA (US); Shidong JIA, Palo Alto, CA (US); Chao DAI, Newark, CA (US)

(21) Appl. No.: 18/950,710

(22) Filed: Nov. 18, 2024

Related U.S. Application Data
(63) Continuation of application No. PCT/US2023/022702, filed on May 18, 2023.
(60) Provisional application No. 63/343,749, filed on May 19, 2022.

(51) Int. Cl. G16B 20/10; C12Q 1/6806; C12Q 1/686; C12Q 1/6869; C12Q 1/6886; G16B 20/20; G16B 40/20; G16H 20/10; G16H 20/40; G16H 50/20; G16H 50/30

(52) U.S. Cl. CPC G16B 20/10; C12Q 1/6806; C12Q 1/686; C12Q 1/6869; C12Q 1/6886; G16B 20/20; G16B 40/20; G16H 20/10; G16H 20/40; G16H 50/20; G16H 50/30; C12Q 2600/118

(57) ABSTRACT

In an aspect, the present disclosure provides a method comprising (a) obtaining or deriving a biological sample from a subject, wherein said subject has cancer, has previously had cancer, or is suspected of having cancer; (b) assaying cell-free deoxyribonucleic acid (cfDNA) molecules obtained or derived from said biological sample, wherein said assaying comprises sequencing at least a portion of said cfDNA molecules or derivatives thereof to produce a set of sequencing reads, wherein said sequencing comprises at least one of whole-exome sequencing (WES) and whole-genome sequencing (WGS); and (c) determining at least one of a tumor mutational burden and a copy number burden of said subject, based at least in part on processing said set of sequencing reads.

### CROSS-REFERENCE

This application is a continuation of International Application No. PCT/US2023/022702, filed May 18, 2023, which claims the benefit of U.S. Provisional Patent Application No. 63/343,749, filed May 19, 2022, each of which is incorporated by reference herein in its entirety.

### BACKGROUND

Cancer is a leading cause of deaths worldwide. Detection of cancer in individuals may be critical for providing treatment and improving patient outcomes. Cancer may be caused by genetic aberration which may lead to unregulated growth of calls. Detection of the genetic aberrations may be important for the detection of cancer. Sequencing of nucleic acids in a sample from a patient may be used to detect genetic aberrations.

### SUMMARY

CDK4/6 inhibition (CDK4/6i) in combination with endocrine therapy (ET) improves survival for patients with hormone receptor-positive (HR+)/HER2-negative (HER2−) metastatic breast cancer (MBC). However, clinical biomarkers to identify patients who may not respond are lacking. We performed genome-wide circulating tumor DNA (ctDNA) analysis to identify features associated with resistance to ET and CDK4/6i.

In an aspect, the present disclosure provides a method comprising: (a) obtaining or deriving a biological sample from a subject, wherein the subject has cancer, has previously had cancer, or is suspected of having cancer; (b) assaying cell-free deoxyribonucleic acid (cfDNA) molecules obtained or derived from the biological sample, wherein the assaying comprises sequencing at least a portion of the cfDNA molecules or derivatives thereof to produce a set of sequencing reads, wherein the sequencing comprises at least one of whole-exome sequencing (WES) and whole-genome sequencing (WGS); and (c) determining at least one of a tumor mutational burden and a copy number burden of the subject, based at least in part on processing the set of sequencing reads.

In some embodiments, the biological sample is selected from the group consisting of: a plasma sample, a serum sample, a buffy coat sample, a urine sample, a saliva sample, a tissue biopsy sample, a pleural fluid sample, a peritoneal fluid sample, an amniotic fluid sample, a cerebrospinal fluid sample, a lymphatic fluid sample, a sweat sample, a tears sample, a semen sample, a derivative thereof, and a combination thereof. In some embodiments, the biological sample comprises the plasma sample. In some embodiments, the biological sample comprises the urine sample.

In some embodiments, the biological sample is a single biological sample of the subject. In some embodiments, the biological sample is a plurality of biological samples of the subject.

In some embodiments, the biological sample is obtained or derived from the subject using an ethylenediaminetetraacetic acid (EDTA) collection tube, a cell-free deoxyribonucleic acid (DNA) collection tube, another blood collection tube, or a circulating tumor cell (CTC) collection tube.

In some embodiments, the method further comprises subjecting the biological sample to conditions that are sufficient to isolate, enrich, or extract the cfDNA molecules.

In some embodiments, the method further comprises fractionating a whole blood sample of the subject to obtain the cfDNA molecules.

In some embodiments, the sequencing further comprises the WES. In some embodiments, the sequencing further comprises the WGS. In some embodiments, the WGS further comprises low-pass WGS. In some embodiments, the sequencing in (b) further comprises next-generation sequencing, low-pass sequencing, targeted sequencing, methylation-aware sequencing, bisulfite sequencing, or a combination thereof. In some embodiments, the sequencing further comprises the methylation-aware sequencing or the bisulfite sequencing.

In some embodiments, (b) further comprises amplifying at least a portion of the cfDNA molecules or derivatives thereof. In some embodiments, the amplifying further comprises polymerase chain reaction (PCR). In some embodiments, the amplifying further comprises isothermal amplification. In some embodiments, (b) further comprises using a microarray.

In some embodiments, the cancer is selected from the group consisting of: breast cancer, lung cancer, prostate cancer, colorectal cancer, melanoma, bladder cancer, non-Hodgkin lymphoma, kidney cancer, endometrial cancer, leukemia, pancreatic cancer, thyroid cancer, liver cancer, and a combination thereof.

In some embodiments, the cancer comprises the breast cancer. In some embodiments, the breast cancer is metastatic breast cancer. In some embodiments, the breast cancer is hormone receptor-positive (HR+) breast cancer. In some embodiments, the breast cancer is HER2-negative (HER2−) breast cancer. In some embodiments, the breast cancer is HR+ and HER2− breast cancer.

In some embodiments, the subject is asymptomatic for the cancer.

In some embodiments, the processing in (c) further comprises using a trained machine learning algorithm.

In some embodiments, the trained machine learning algorithm is trained using at least 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 independent training samples.

In some embodiments, the trained machine learning algorithm is trained using a first set of independent training samples associated with a presence of the cancer and a second set of independent training samples associated with an absence of the cancer.

In some embodiments, the trained machine learning algorithm is trained using a first set of independent training samples associated with presence of a relapse or recurrence of the cancer and a second set of independent training samples associated with absence of relapse or recurrence of the cancer.

In some embodiments, the trained machine learning algorithm is trained using a first set of independent training samples associated with presence of a drug treatment or resistance to drug treatment of the cancer and a second set of independent training samples associated with absence of a drug treatment or resistance to drug treatment of the cancer. In some embodiments, the trained machine learning algorithm further comprises an unsupervised machine learning algorithm. In some embodiments, the trained machine learning algorithm further comprises a supervised machine learning algorithm. In some embodiments, the supervised machine learning algorithm further comprises a deep learning algorithm, a support vector machine (SVM), a neural network, or a Random Forest.

In some embodiments, (c) further comprises using the trained machine learning algorithm or another trained machine learning algorithm to process a set of clinical health data of the subject.

In some embodiments, the method further comprises determining a relapse or recurrence of the cancer of the subject, based at least in part on the at least one of the tumor mutational burden and the copy number burden of the subject.

In some embodiments, the method further comprises determining a relapse or recurrence of the cancer of the subject, based at least in part on the at least one of the tumor mutational burden and the copy number burden of the subject being at least about 10%, at least about 20%, at least about 30%, at least about 40%, at least about 50%, at least about 60%, at least about 70%, at least about 80%, or at least about 90%.

In some embodiments, the method further comprises determining a relapse or recurrence of the cancer of the subject, based at least in part on both of the tumor mutational burden and the copy number burden of the subject being at least about 10%, at least about 20%, at least about 30%, at least about 40%, at least about 50%, at least about 60%, at least about 70%, at least about 80%, or at least about 90%.

In some embodiments, the method further comprises determining the relapse or the recurrence with an accuracy of at least about 60%, at least about 70%, at least about 75%, at least about 80%, at least about 85%, at least about 90%, at least about 95%, at least about 98%, or at least about 99%.

In some embodiments, the method further comprises determining the relapse or the recurrence with a sensitivity of at least about 60%, at least about 70%, at least about 75%, at least about 80%, at least about 85%, at least about 90%, at least about 95%, at least about 98%, or at least about 99%.

In some embodiments, the method further comprises determining the relapse or the recurrence with a specificity of at least about 60%, at least about 70%, at least about 75%, at least about 80%, at least about 85%, at least about 90%, at least about 95%, at least about 98%, or at least about 99%.

In some embodiments, the method further comprises determining the relapse or the recurrence with a positive predictive value of at least about 60%, at least about 70%, at least about 75%, at least about 80%, at least about 85%, at least about 90%, at least about 95%, at least about 98%, or at least about 99%.

In some embodiments, the method further comprises determining the relapse or the recurrence with a negative predictive value of at least about 60%, at least about 70%, at least about 75%, at least about 80%, at least about 85%, at least about 90%, at least about 95%, at least about 98%, or at least about 99%.

In some embodiments, the method further comprises determining a resistance of the cancer to a drug treatment, based at least in part on the at least one of the tumor mutational burden and the copy number burden of the subject.

In some embodiments, the method further comprises determining the resistance of the cancer to the drug treatment, based at least in part on the at least one of the tumor mutational burden and the copy number burden of the subject being at least about 10%, at least about 20%, at least about 30%, at least about 40%, at least about 50%, at least about 60%, at least about 70%, at least about 80%, or at least about 90%.

In some embodiments, the method further comprises determining the resistance of the cancer to the drug treatment, based at least in part on both of the tumor mutational burden and the copy number burden of the subject being at least about 10%, at least about 20%, at least about 30%, at least about 40%, at least about 50%, at least about 60%, at least about 70%, at least about 80%, or at least about 90%.

In some embodiments, the method further comprises determining the resistance with an accuracy of at least about 60%, at least about 70%, at least about 75%, at least about 80%, at least about 85%, at least about 90%, at least about 95%, at least about 98%, or at least about 99%.

In some embodiments, the method further comprises determining the resistance with a sensitivity of at least about 60%, at least about 70%, at least about 75%, at least about 80%, at least about 85%, at least about 90%, at least about 95%, at least about 98%, or at least about 99%.

In some embodiments, the method further comprises determining the resistance with a specificity of at least about 60%, at least about 70%, at least about 75%, at least about 80%, at least about 85%, at least about 90%, at least about 95%, at least about 98%, or at least about 99%.

In some embodiments, the method further comprises determining the resistance with a positive predictive value of at least about 60%, at least about 70%, at least about 75%, at least about 80%, at least about 85%, at least about 90%, at least about 95%, at least about 98%, or at least about 99%.

In some embodiments, the method further comprises determining the resistance with a negative predictive value of at least about 60%, at least about 70%, at least about 75%, at least about 80%, at least about 85%, at least about 90%, at least about 95%, at least about 98%, or at least about 99%.

In some embodiments, the method further comprises determining a prognosis of the cancer of the subject, based at least in part on the at least one of the tumor mutational burden and the copy number burden of the subject. In some embodiments, the method further comprises determining a prognosis of the cancer of the subject, based at least in part on both of the tumor mutational burden and the copy number burden of the subject. In some embodiments, the prognosis comprises a likelihood of progression-free survival, a length of time for progression-free survival, a likelihood of overall survival, a length of time for overall survival, or a combination thereof.

In some embodiments, (a) further comprises obtaining or deriving the biological sample from the subject, (i) prior to the subject receiving a clinical intervention for the cancer, (ii) while the subject is receiving a clinical intervention for the cancer, (iii) subsequent to the subject receiving a clinical intervention for the cancer, or a combination thereof. In some embodiments, the clinical intervention is selected from the group consisting of: surgical resection, chemotherapy, radiotherapy, immunotherapy, cell therapy, adjuvant therapy, neoadjuvant therapy, androgen deprivation therapy, and a combination thereof.

In some embodiments, the method further comprises selecting a clinical intervention for the subject, based at least in part on the at least one of the tumor mutational burden and the copy number burden of the subject. In some embodiments, the method further comprises selecting the clinical intervention for the subject, based at least in part on both of the tumor mutational burden and the copy number burden of the subject. In some embodiments, the clinical intervention is selected from a plurality of clinical interventions.

In some embodiments, the clinical intervention is selected from the group consisting of: surgical resection, chemotherapy, radiotherapy, immunotherapy, endocrine therapy, adjuvant therapy, neoadjuvant therapy, androgen deprivation therapy, and a combination thereof. In some embodiments, the clinical intervention comprises a CDK4/6 inhibitor. In some embodiments, the CDK4/6 inhibitor comprises palbociclib. In some embodiments, the clinical intervention comprises the endocrine therapy. In some embodiments, the endocrine therapy comprises letrozole or fulvestrant. In some embodiments, the clinical intervention comprises the endocrine therapy and a CDK4/6 inhibitor.

In some embodiments, the method further comprises administering the clinical intervention to the subject.

In some embodiments, the set of sequencing reads comprises quantitative measures of a set of cancer-associated genomic loci. In some embodiments, the set of cancer-associated genomic loci comprises one or more members selected from the group consisting of genes listed in Table 3, genes listed in Table 4, genes listed in Table 6, and genes listed in Table 7. In some embodiments, the set of cancer-associated genomic loci comprises at least 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 members selected from the group consisting of genes listed in Table 3, genes listed in Table 4, genes listed in Table 6, and genes listed in Table 7. In some embodiments, the set of cancer-associated genomic loci comprises one or more members selected from the group consisting of genes listed in Table 3. In some embodiments, the set of cancer-associated genomic loci comprises one or more members selected from the group consisting of genes listed in Table 4. In some embodiments, the set of cancer-associated genomic loci comprises one or more members selected from the group consisting of genes listed in Table 6. In some embodiments, the set of cancer-associated genomic loci comprises one or more members selected from the group consisting of genes listed in Table 7.

In some embodiments, (b) further comprises using nucleic acid primers or probes configured to selectively enrich the biological sample for DNA molecules corresponding to a set of genomic loci. In some embodiments, the nucleic acid primers or probes have sequence complementarity with at least a portion of nucleic acid sequences of the set of genomic loci. In some embodiments, the nucleic acid primers or probes comprise at least 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 different nucleic acid primers or probes.

In some embodiments, the method further comprises monitoring at least one of the tumor mutational burden and the copy number burden of the subject, wherein the monitoring comprises assessing, at each of a plurality of time points, the at least one of the tumor mutational burden and the copy number burden of the subject. In some embodiments, a difference in the assessing of the at least one of the tumor mutational burden and the copy number burden of the subject among the plurality of time points is indicative of one or more clinical indications selected from the group consisting of: (i) a diagnosis of the cancer, (ii) a prognosis of the cancer, and (iii) an efficacy or non-efficacy of a clinical intervention for treating the cancer of the subject.

In some embodiments, the processing in (c) further comprises detecting tumor-associated alterations selected from the group consisting of: copy number alterations (CNAs), copy number losses (CNLs), single nucleotide variants (SNVs), insertions or deletions (indels), and rearrangements.

In some embodiments, the method further comprises filtering at least a subset of the set of sequencing reads based on a quality score.

In some embodiments, the method further comprises performing error correction on the set of sequencing reads using sample barcodes or molecular barcodes attached to at least one of the cfDNA molecules.

In some embodiments, the method further comprises performing at least one of single-stranded consensus calling and double-stranded consensus calling on the set of sequencing reads, thereby suppressing sequencing and PCR errors in the set of sequencing reads.

In some embodiments, the method further comprises determining a mutant allele frequency of a set of somatic mutations.

In another aspect, the present disclosure provides a system comprising one or more computer processors and computer memory coupled thereto, the computer memory comprising machine executable code that, upon execution by the one or more computer processors, implements a method comprising: (a) obtaining or deriving a biological sample from a subject, wherein the subject has cancer, has previously had cancer, or is suspected of having cancer; (b) assaying cell-free deoxyribonucleic acid (cfDNA) molecules obtained or derived from the biological sample, wherein the assaying comprises sequencing at least a portion of the cfDNA molecules or derivatives thereof to produce a set of sequencing reads, wherein the sequencing comprises at least one of whole-exome sequencing (WES) and whole-genome sequencing (WGS); and (c) determining at least one of a tumor mutational burden and a copy number burden of the subject, based at least in part on processing the set of sequencing reads.

In another aspect, the present disclosure provides a non-transitory computer readable medium comprising machine executable code that, upon execution by one or more computer processors, implements a method comprising: (a) obtaining or deriving a biological sample from a subject, wherein the subject has cancer, has previously had cancer, or is suspected of having cancer; (b) assaying cell-free deoxyribonucleic acid (cfDNA) molecules obtained or derived from the biological sample, wherein the assaying comprises sequencing at least a portion of the cfDNA molecules or derivatives thereof to produce a set of sequencing reads, wherein the sequencing comprises at least one of whole-exome sequencing (WES) and whole-genome sequencing (WGS); and (c) determining at least one of a tumor mutational burden and a copy number burden of the subject, based at least in part on processing the set of sequencing reads.

Also provided herein are systems and methods for detection of the presence or absence of cancer in a subject. The systems and methods provided herein comprises assaying polynucleotides to identify biomarkers of cancers in a subject. Detection of a type of cancer or the specific biomarkers for a given cancer may allow an effective treatment to be provided to an individual and may result in improved outcomes. For multiple types of cancer, the particular biomarkers that indicate a particular cancer type (or subtype) may be used to identify a prognosis for an individual suffering from the cancer. In order to provide accurate detection and prognosis for a cancer, multiple analytes may be examined. By analyzing an increased number of analytes (and sets of biomarkers from the analytes), the detection of a cancer (or cancer parameter) may be improved and may allow for the recommendation of an effective treatment, and may also allow for the prognosis to be more accurate.

In an aspect, the present disclosure provides a method for detecting a presence or an absence of cancer in a subject, comprising: (a) assaying cell-free deoxyribonucleic acid (cfDNA) molecules and cell-free ribonucleic (cfRNA) molecules from a biological sample obtained or derived from the subject to detect a first set of biomarkers from the cfDNA molecules and a second set of biomarkers from the cfRNA molecules; and (b) computer processing the first set of biomarkers and the second set of biomarkers to detect the presence or the absence of the cancer in the subject.

In some embodiments, the biological sample is selected from the group consisting of: a cell-free deoxyribonucleic acid (cfDNA) sample, a cell-free ribonucleic acid (cfRNA) sample, a plasma sample, a serum sample, a buffy coat sample, a peripheral blood mononuclear cell (PBMC) sample, a red blood cell sample, a urine sample, a saliva sample, tissue biopsy, pleural fluid sample, peritoneal fluid sample, amniotic fluid sample, cerebroshinal fluid sample, lymphatic fluid sample, sweat sample, tear sample, semen sample, or any derivative thereof, and any combination thereof. In some embodiments, the biological sample comprises the plasma sample. In some embodiments, the biological sample comprises the urine sample.

In some embodiments, the cfDNA molecules and the cfRNA molecules are obtained or derived from a single biological sample of the subject. In some embodiments, the cfDNA molecules and the cfRNA molecules are obtained or derived from different biological samples of the subject.

In some embodiments, the biological sample is obtained or derived from the subject using an ethylenediaminetetraacetic acid (EDTA) collection tube, a cell-free RNA collection tube, or a cell-free deoxyribonucleic acid (DNA) collection tube, other blood collection tube, and CTC collection tubes.

In some embodiments, (a) comprises subjecting the biological sample to conditions that are sufficient to isolate, enrich, or extract the cfDNA molecules and the set of cfRNA molecules.

In some embodiments, the method further comprises fractionating a whole blood sample of the subject to obtain the cfDNA molecules and the cfRNA molecules.

In some embodiments, at least one of the cfDNA molecules and the cfRNA molecules are assayed using nucleic acid sequencing to produce nucleic acid sequencing reads. In some embodiments, the cfDNA molecules are assayed using DNA sequencing. In some embodiments, the DNA sequencing is selected from the group consisting of: next-generation sequencing, whole genome sequencing, low-pass sequencing, targeted sequencing, methylation-aware sequencing, enzymatic methylation sequencing, bisulfite methylation sequencing, and a combination thereof. In some embodiments, the DNA sequencing comprises low-pass whole genome sequencing. In some embodiments, the DNA sequencing comprises whole exome sequencing. In some embodiments, the DNA sequencing comprises methylation aware sequencing, enzymatic methylation sequencing or bisulfite methylation sequencing.

In some embodiments, the cfRNA molecules are assayed using RNA sequencing. In some embodiments, the RNA sequencing is selected from the group consisting of: next-generation sequencing, transcriptome sequencing, mRNA-seq, totalRNA-seq, smallRNA-seq, exosome sequencing, and a combination thereof. In some embodiments, the RNA sequencing comprises reverse transcribing the cfRNA molecules into complementary DNA (cDNA) molecules, and performing DNA sequencing on the cDNA molecules.

In some embodiments, the nucleic acid sequencing comprises nucleic acid amplification. In some embodiments, the nucleic acid amplification comprises polymerase chain reaction (PCR) or isothermal amplification. In some embodiments, the nucleic acid sequencing comprises use of substantially simultaneous reverse transcription (RT) and polymerase chain reaction (PCR).

In some embodiments, at least one of the cfDNA molecules and the cfRNA molecules are assayed using a polymerase chain reaction (PCR) assay, microarray, or a isothermal amplification.

In some embodiments, the cancer is selected from the group consisting of: breast cancer, lung cancer, prostate cancer, colorectal cancer, melanoma, bladder cancer, non-Hodgkin lymphoma, kidney cancer, endometrial cancer, leukemia, pancreatic cancer, thyroid cancer, and liver cancer, and any combination thereof. In some embodiments, the cancer comprises the prostate cancer. In some embodiments, the prostate cancer is selected from the group consisting of: hormone sensitive prostate cancer (HSPC), castrate-resistant prostate cancer (CRPC), metastatic prostate cancer, and a combination thereof. In some embodiments, the subject is asymptomatic for the cancer. In some embodiments, the cancer comprises the breast cancer. In some embodiments, the cancer comprises bladder cancer.

In some embodiments, (b) comprises processing the first set of biomarkers and the second set of biomarkers using a trained algorithm. In some embodiments, the trained algorithm is trained using at least 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 independent training samples associated with a presence or an absence of the cancer. In some embodiments, the trained algorithm is trained using at least 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 independent training samples associated with a relapse of cancer. In some embodiments, the trained algorithm is trained using at least 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 independent training samples associated with a drug treatment or resistance to the drug treatment.

In some embodiments, the trained algorithm is trained using a first set of independent training samples associated with a presence of the cancer and a second set of independent training samples associated with an absence of the cancer. In some embodiments, the trained algorithm is trained using a first set of independent training samples associated with a presence of the cancer and a second set of independent training samples associated with a relapse of cancer. In some embodiments, the trained algorithm is trained using a first set of independent training samples associated with a presence of the cancer and a second set of independent training samples associated with a drug treatment or resistance to the drug treatment.

In some embodiments, the method further comprises using the trained algorithm or another trained algorithm to process a set of clinical health data of the subject to determine the presence or the absence of the cancer. In some embodiments, the method further comprises using the trained algorithm or another trained algorithm to process a set of clinical health data of the subject to determine a relapse of cancer. In some embodiments, the method further comprises using the trained algorithm or another trained algorithm to process a set of clinical health data of the subject to determine a drug treatment or resistance to the drug treatment.

In some embodiments, the trained algorithm comprises an un-supervised machine learning algorithm. In some embodiments, the trained algorithm comprises a supervised machine learning algorithm. In some embodiments, the supervised machine learning algorithm comprises a deep learning algorithm, a support vector machine (SVM), a neural network, or a Random Forest.

In some embodiments, (b) comprises detecting the presence or the absence of the cancer in the subject at an accuracy of at least about 60%, at least about 70%, at least about 75%, at least about 80%, at least about 85%, at least about 90%, at least about 95%, at least about 98%, or at least about 99%.

In some embodiments, (b) comprises detecting the presence or the absence of the cancer in the subject at a sensitivity of at least about 60%, at least about 70%, at least about 75%, at least about 80%, at least about 85%, at least about 90%, at least about 95%, at least about 98%, or at least about 99%.

In some embodiments, (b) comprises detecting the presence or the absence of the cancer in the subject at a specificity of at least about 60%, at least about 70%, at least about 75%, at least about 80%, at least about 85%, at least about 90%, at least about 95%, at least about 98%, or at least about 99%.

In some embodiments, (b) comprises detecting the presence or the absence of the cancer in the subject at a positive predictive value of at least about 60%, at least about 70%, at least about 75%, at least about 80%, at least about 85%, at least about 90%, at least about 95%, at least about 98%, or at least about 99%.

In some embodiments, (b) comprises detecting the presence or the absence of the cancer in the subject at a negative predictive value of at least about 60%, at least about 70%, at least about 75%, at least about 80%, at least about 85%, at least about 90%, at least about 95%, at least about 98%, or at least about 99%.

In some embodiments, the biological sample is obtained or derived from the subject prior to the subject receiving a therapy for the cancer. In some embodiments, the biological sample is obtained or derived from the subject during a therapy for the cancer. In some embodiments, the biological sample is obtained or derived from the subject after receiving a therapy for the cancer.

In some embodiments, the therapy is selected from the group consisting of: surgical resection, chemotherapy, radiotherapy, immunotherapy, cell therapy, adjuvant therapy, neoadjuvant therapy, androgen deprivation therapy, and a combination thereof.

In some embodiments, the method further comprises identifying a clinical intervention for the subject based at least in part on the detected presence or the absence of the cancer. In some embodiments, the clinical intervention is selected from a plurality of clinical interventions. In some embodiments, the clinical intervention is selected from the group consisting of: surgical resection, chemotherapy, radiotherapy, immunotherapy, adjuvant therapy, neoadjuvant therapy, androgen deprivation therapy, and a combination thereof. In some embodiments, the method further comprises administering the clinical intervention to the subject.

In some embodiments, the first set of biomarkers comprises quantitative measures of a first set of cancer-associated genomic loci. In some embodiments, the first set of cancer-associated genomic loci comprises one or more members selected from the group consisting of genes listed in Table 1. In some embodiments, the first set of cancer-associated genomic loci comprises 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 members selected from the group consisting of genes listed in Table 1. In some embodiments, the first set of cancer-associated genomic loci comprises PTEN, TP53 or RB1. In some embodiments, the first set of cancer-associated genomic loci comprises PTEN, TP53 and RB1. In some embodiments, the first set of cancer-associated genomic loci comprises PTEN. In some embodiments, the first set of cancer-associated genomic loci comprises FGFR3 or ERBB2.

In some embodiments, the second set of biomarkers comprises quantitative measures of a second set of cancer-associated genomic loci. In some embodiments, the second set of cancer-associated genomic loci comprises one or more members selected from the group consisting of genes listed in Table 2. In some embodiments, the second set of cancer-associated genomic loci comprises 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 members selected from the group consisting of genes listed in Table 2.

In some embodiments, the method further comprises using probes configured to selectively enrich the biological sample for nucleic acid molecules corresponding to a set of genomic loci. In some embodiments, the probes are nucleic acid primers. In some embodiments, the probes have sequence complementarity with at least a portion of nucleic acid sequences of the set of genomic loci. In some embodiments, the probes comprise at least 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 different probes.

In some embodiments, the method further comprises determining a likelihood of the determination of the presence or the absence of the cancer in the subject.

In some embodiments, the method further comprises monitoring the presence or the absence of the cancer in the subject, wherein the monitoring comprises assessing the presence or the absence of the cancer in the subject at each of a plurality of time points.

In some embodiments, a difference in the assessment of the presence or the absence of the cancer in the subject among the plurality of time points is indicative of one or more clinical indications selected from the group consisting of: (i) a diagnosis of the cancer, (ii) a prognosis of the cancer, and (iii) an efficacy or non-efficacy of a course of treatment for treating the cancer of the subject. In some embodiments, the prognosis comprises an expected progression-free survival (PFS) or overall survival (OS).

In some embodiments, the method further comprises assaying germline DNA (gDNA) molecules obtained or derived from the subject to detect a third set of biomarkers, and computer processing the third set of biomarkers to detect the presence or the absence of the cancer in the subject.

In some embodiments, the first set of biomarkers from the cfDNA molecules comprise tumor-associated alterations selected from the group consisting of: copy number alterations (CNAs), copy number losses (CNLs), loss of heterozygosity (LOH), single nucleotide variants (SNVs), insertions or deletions (indels), rearrangements, and epigenetic changes such as methylation. In some embodiments, the first set of biomarkers from the cfDNA molecules comprise copy number variation. In some embodiments, the first set of biomarkers from the cfDNA molecules comprise copy number losses. In some embodiments, the first set of biomarkers from the cfDNA molecules comprise single nucleotide variants.

In some embodiments, the second set of biomarkers from the cfRNA molecules comprise tumor-associated alterations selected from the group consisting of: alternative splicing variants, fusions, single nucleotide variants (SNVs), and insertions or deletions (indels).

In some embodiments, the method further comprises filtering at least a subset of the nucleic acid sequencing reads based on a quality score.

In some embodiments, the method further comprises performing error correction on the nucleic acid sequencing reads using sample barcodes or molecular barcodes attached to at least one of the cfDNA molecules and the cfRNA molecules.

In some embodiments, the method further comprises performing at least one of single-stranded consensus calling and double-stranded consensus calling on the nucleic acid sequencing reads, thereby suppressing sequencing and PCR errors in the nucleic acid sequencing reads.

In some embodiments, the method further comprises determining, among the first set of biomarkers, a mutant allele frequency of a set of somatic mutations. In some embodiment, the method further comprises determining a blood copy number burden based on copy number alterations or copy number losses of the first set of biomarkers.

In some embodiments, the method further comprises determining a circulating tumor DNA (ctDNA) fraction of the cancer of the subject based at least in part on the set of mutant allele frequencies.

In some embodiments, the method further comprises determining a plasma tumor mutational burden (pTMB) of the cancer of the subject based at least in part on the set of mutant allele frequencies.

In some embodiments, the method further comprises determining a plasma tumor mutational burden (pTMB) of the cancer of the subject based at least in part on the set of mutant allele frequencies comprising microsatellites.

In some embodiments, the method further comprises determining an abnormality score of the cancer of the subject based at least in part on the set of mutant allele frequencies.

In some embodiments, the method further comprises determining a methylation related score of the cancer of the subject based at least in part on the set of mutant allele frequencies.

In another aspect, the present disclosure provides a method for detecting a presence or an absence of prostate cancer in a subject, comprising: (a) assaying cell-free deoxyribonucleic acid (cfDNA) molecules and germline DNA (gDNA) molecules from a biological sample obtained or derived from the subject to detect a first set of biomarkers from the cfDNA molecules and a second set of biomarkers from the gRNA molecules, wherein at least one of the first set of biomarkers and the second set of biomarkers comprises an androgen receptor (AR) alteration; and (b) computer processing the first set of biomarkers and the second set of biomarkers to detect the presence or the absence of the prostate cancer in the subject.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIGS. 1A-1C provide an overview of an example study design, including: study schema (FIG. 1A), sample collection timepoints for PredicineCNB™ and PredicineWES⁺™ profiling (FIG. 1B), and next-generation sequencing process (FIG. 1C).

FIGS. 2A-2H show that high bTMB at baseline is associated with poor patient outcomes. FIG. 2A shows a distribution of bTMB scores across 50 baseline patient samples sequenced by PredicineWES+™.

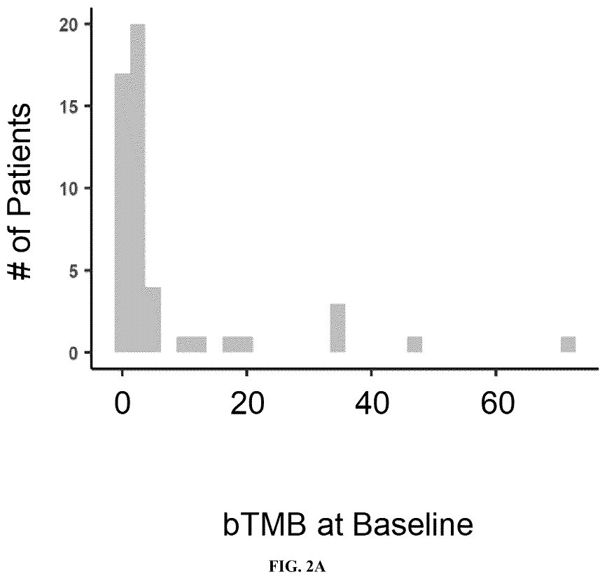

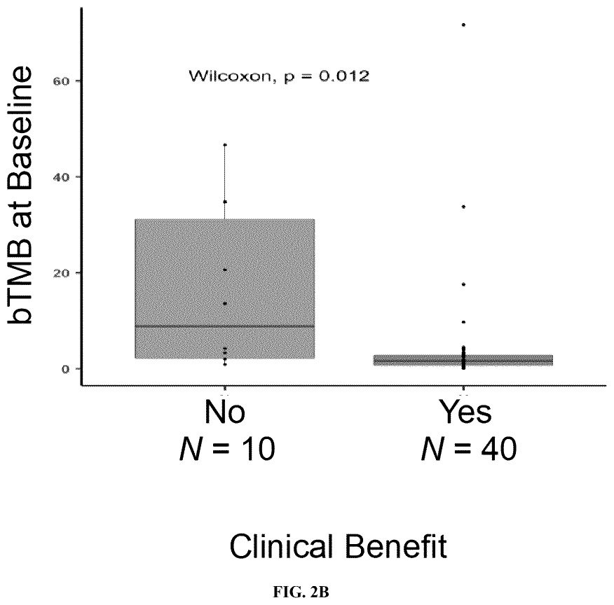

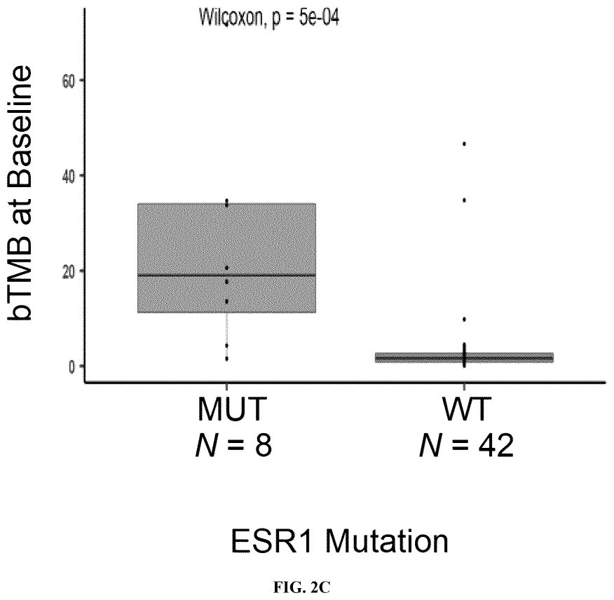

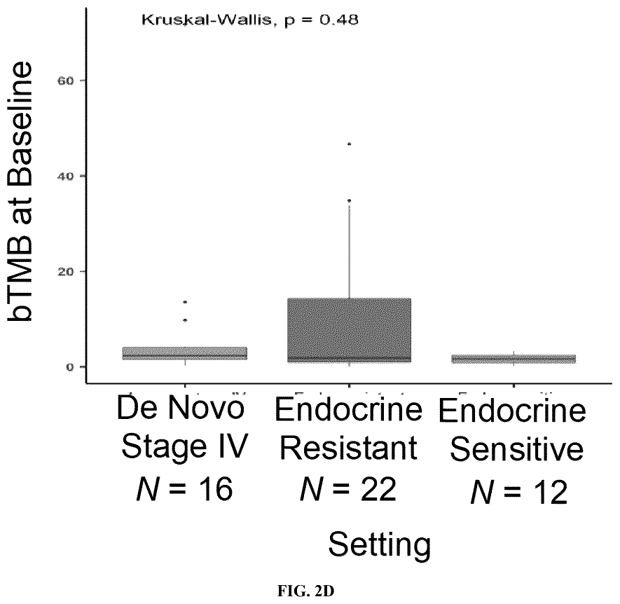

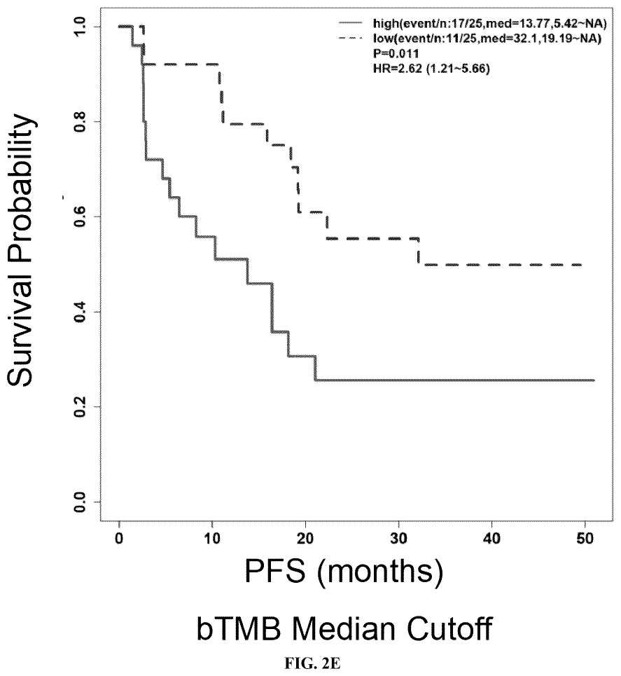

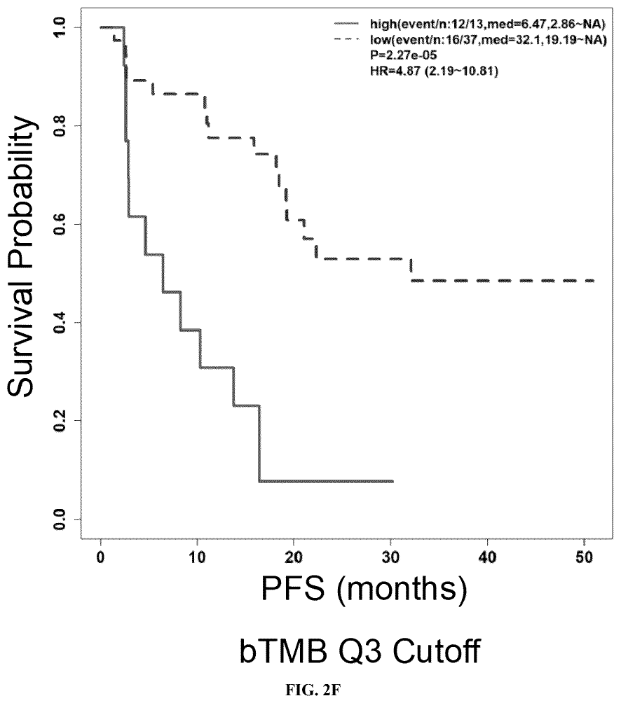

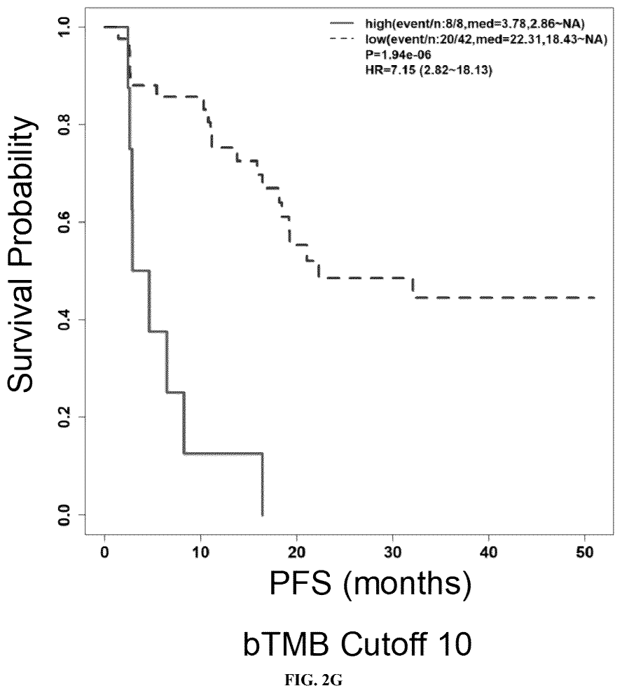

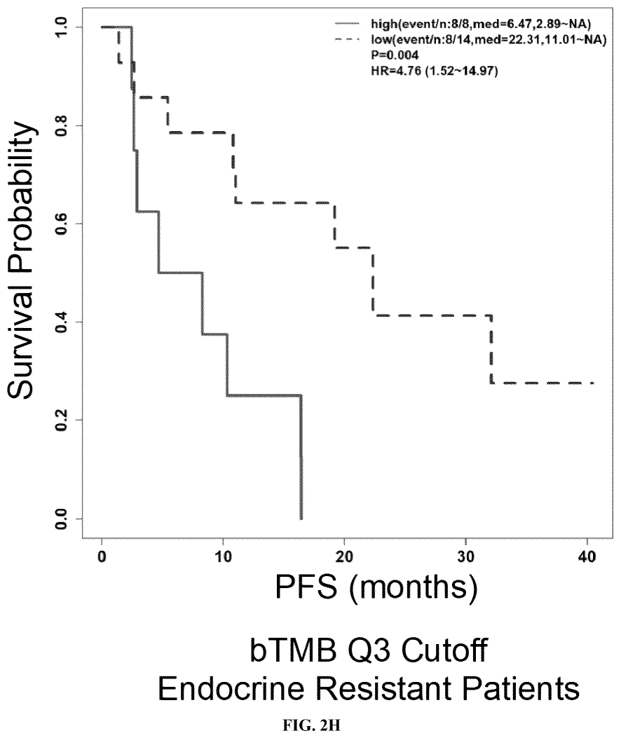

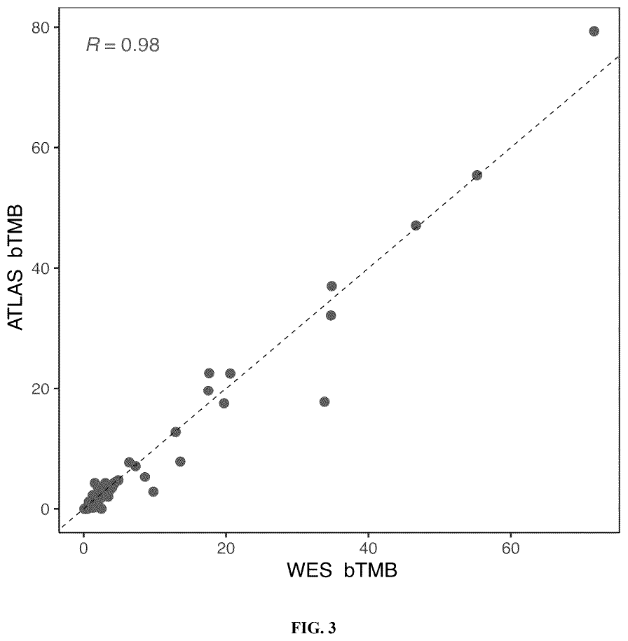

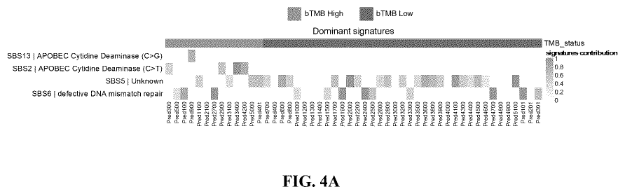

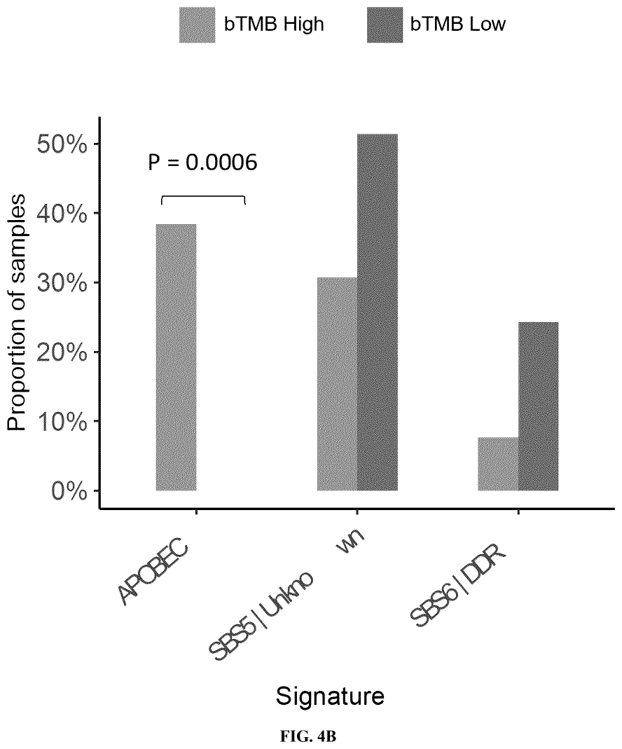

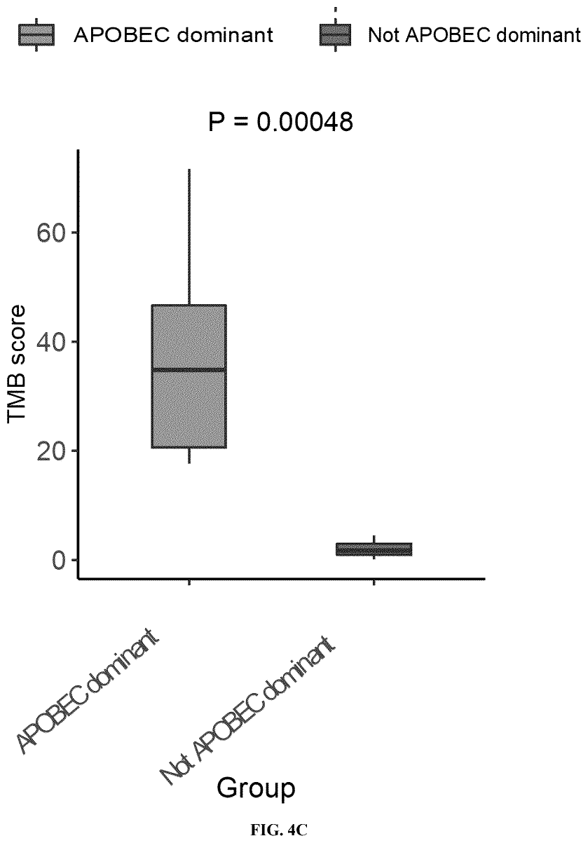

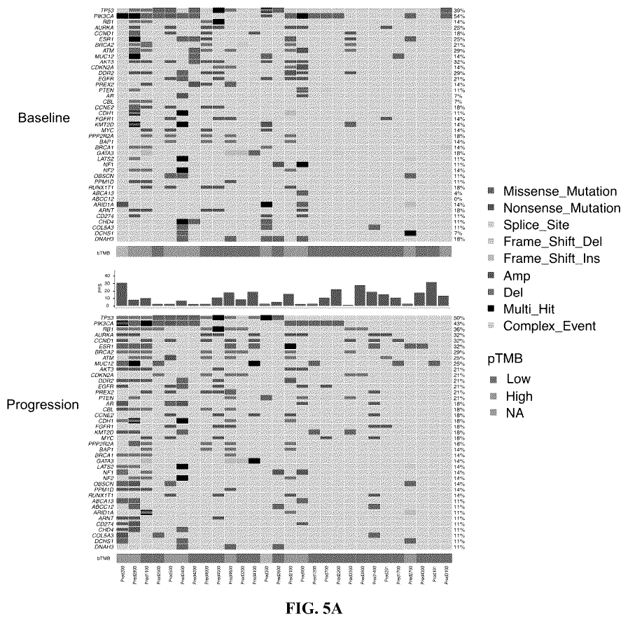

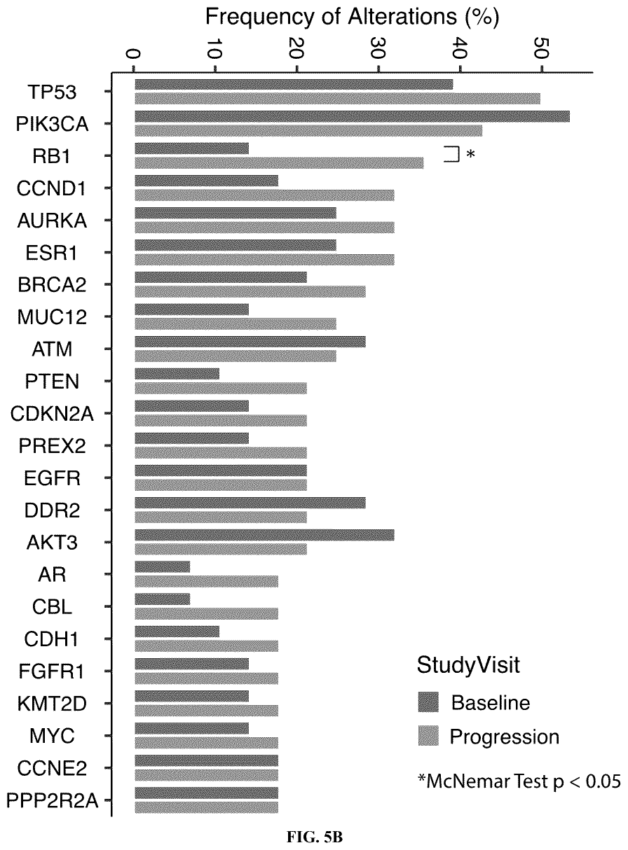

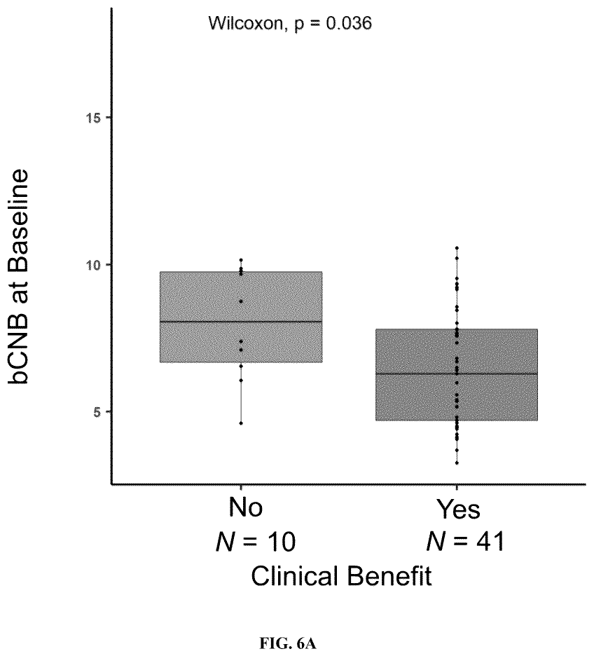

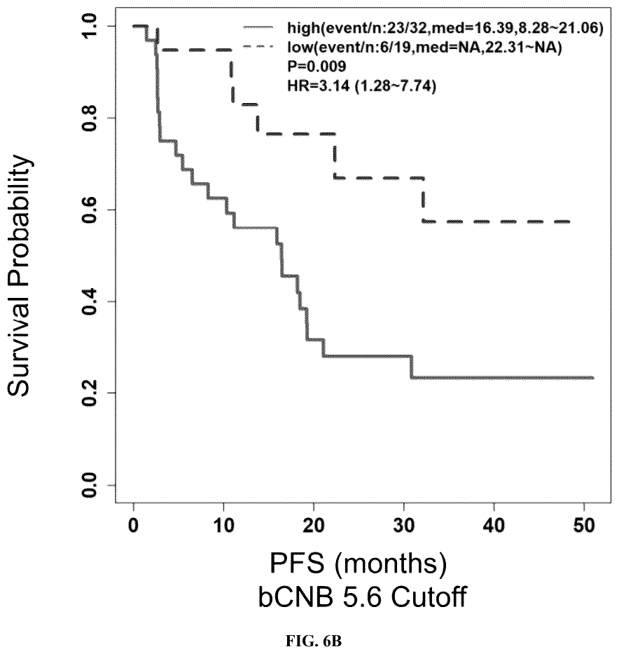

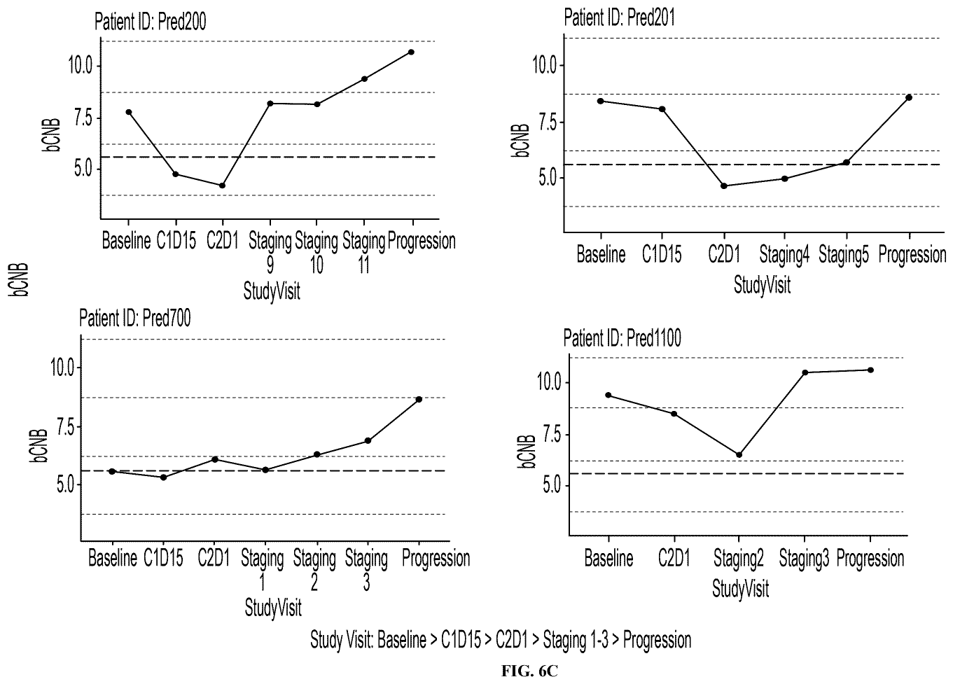

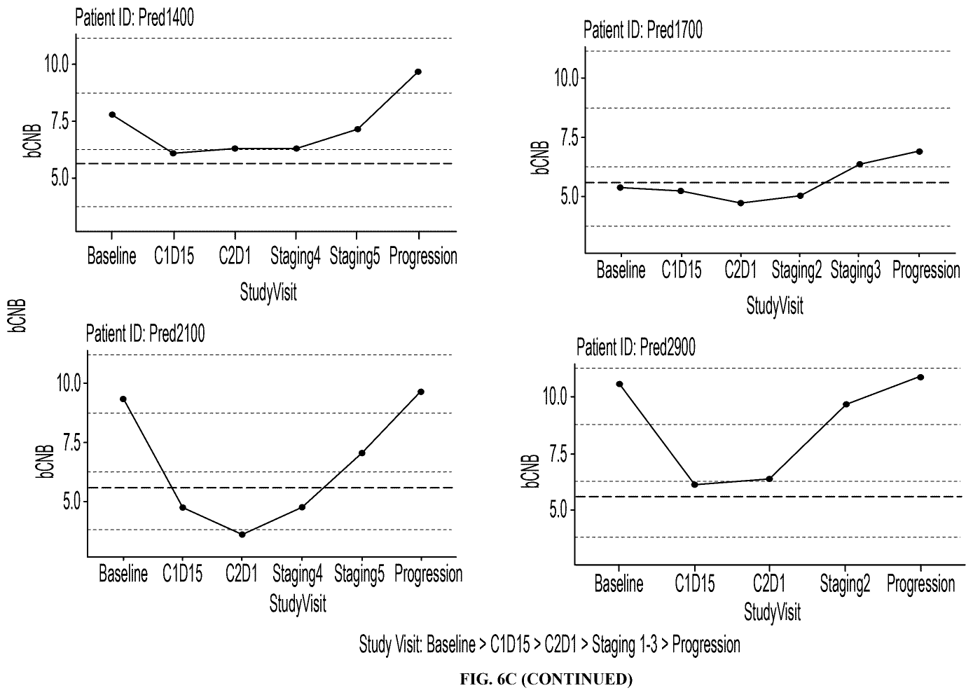

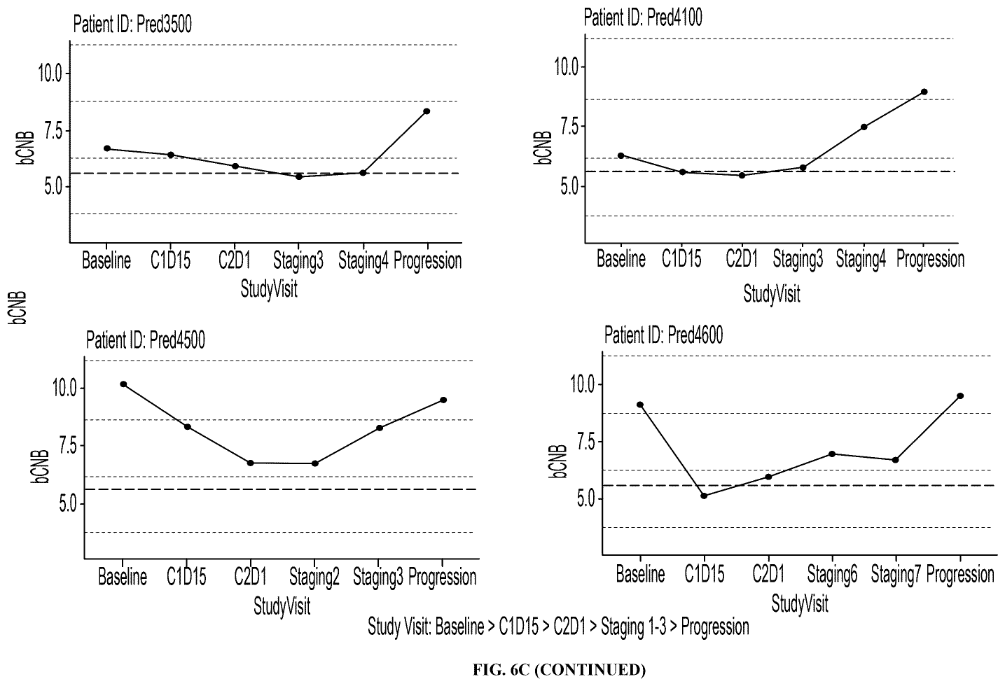

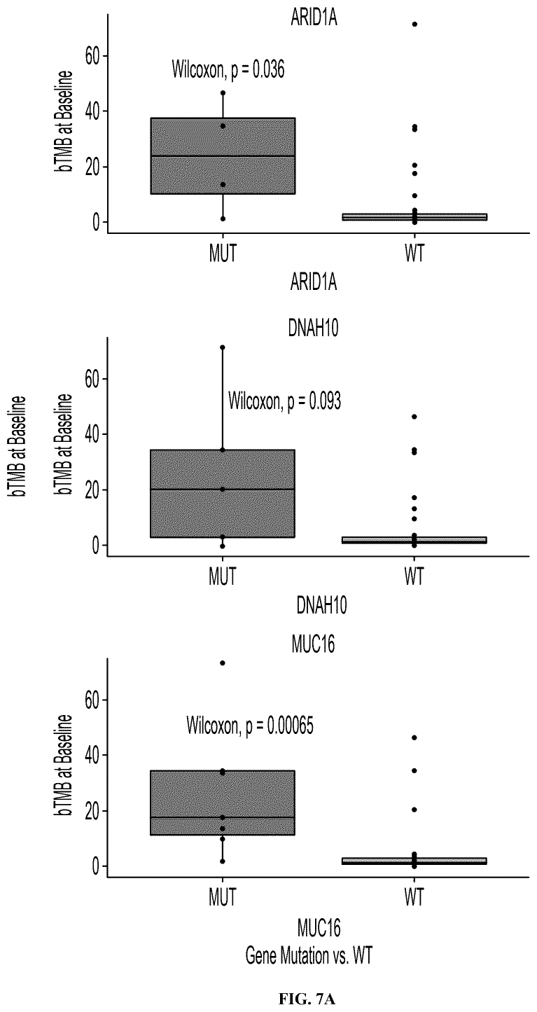

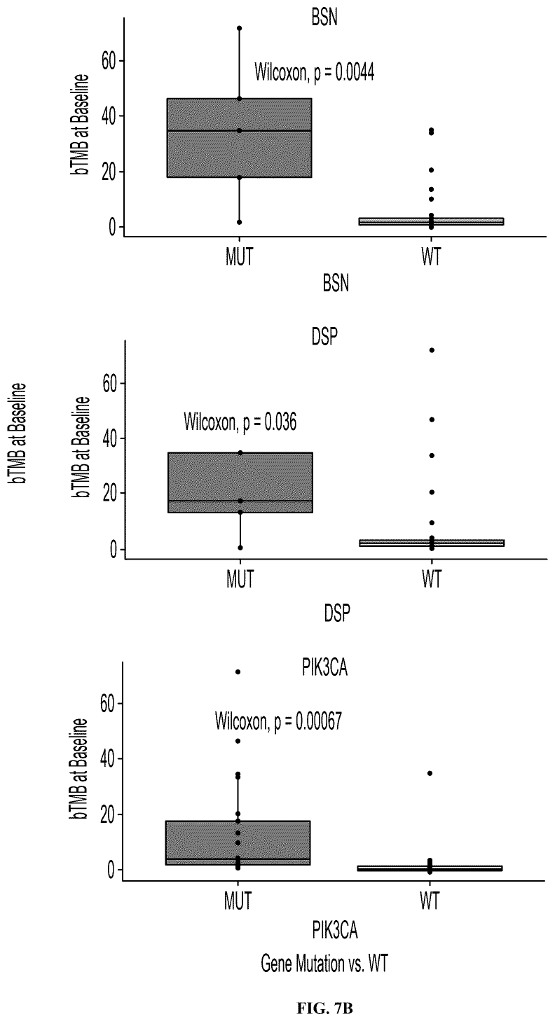

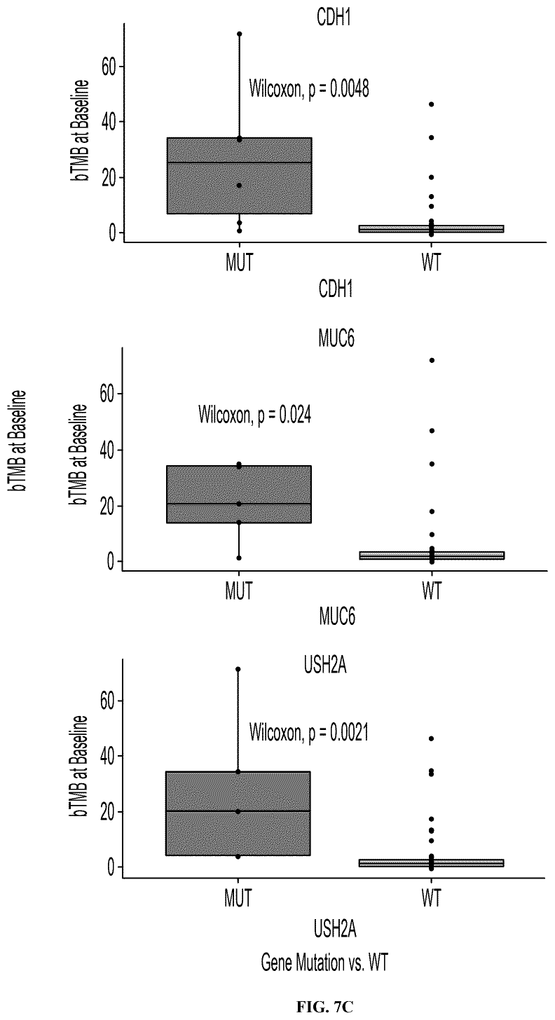

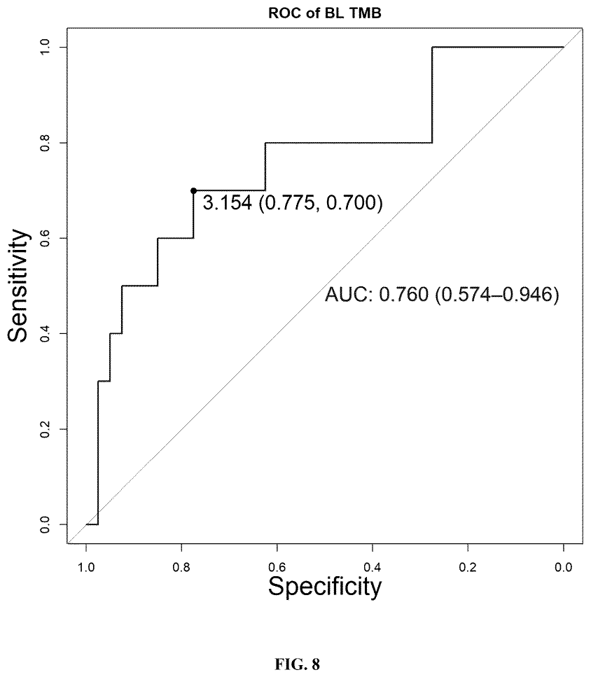

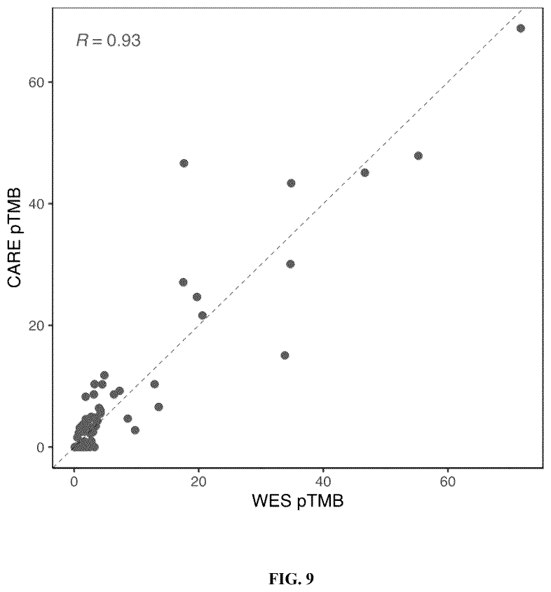

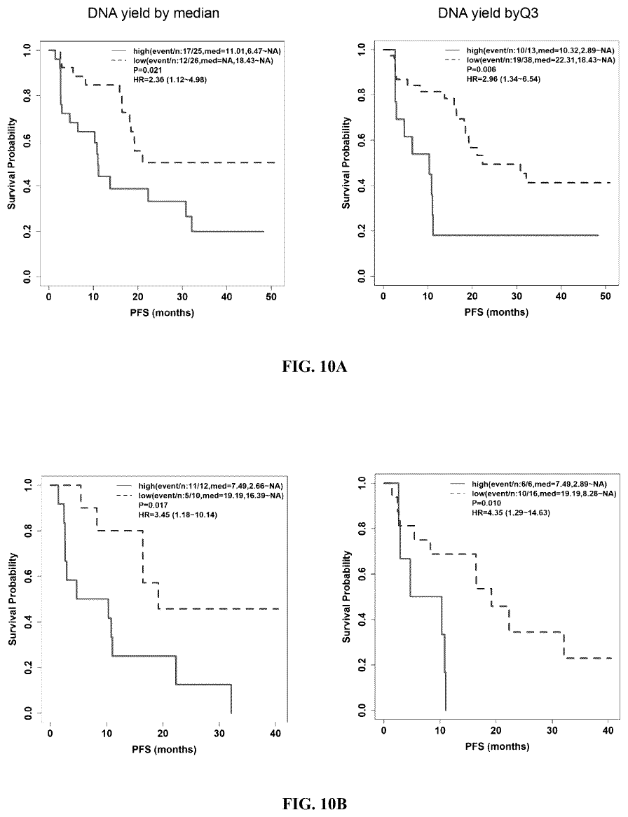

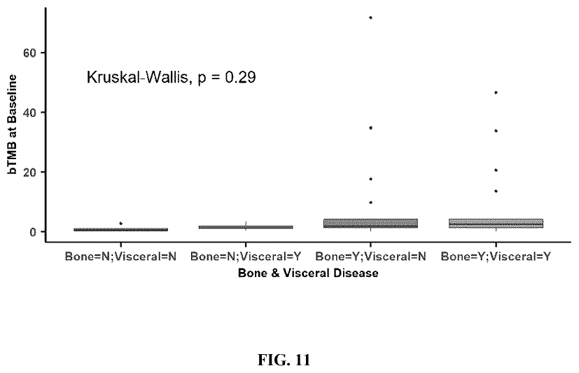

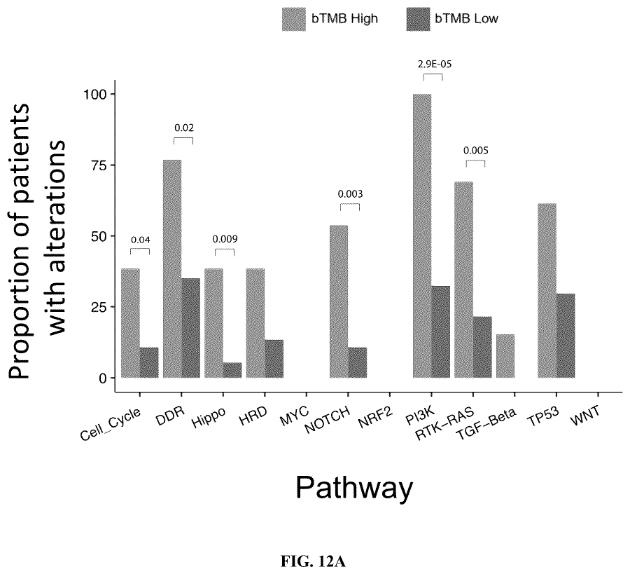

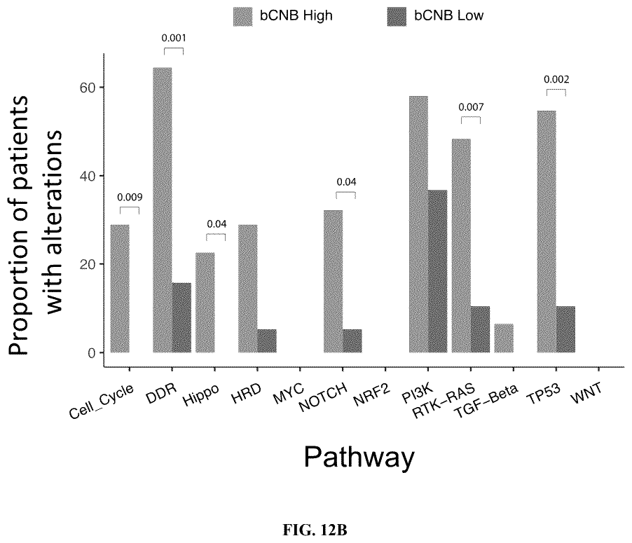

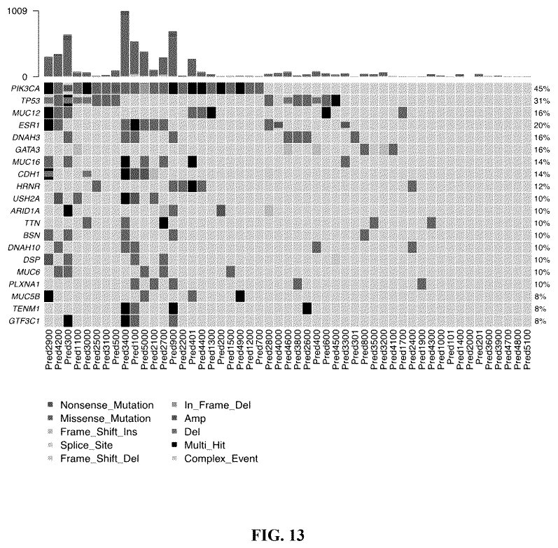

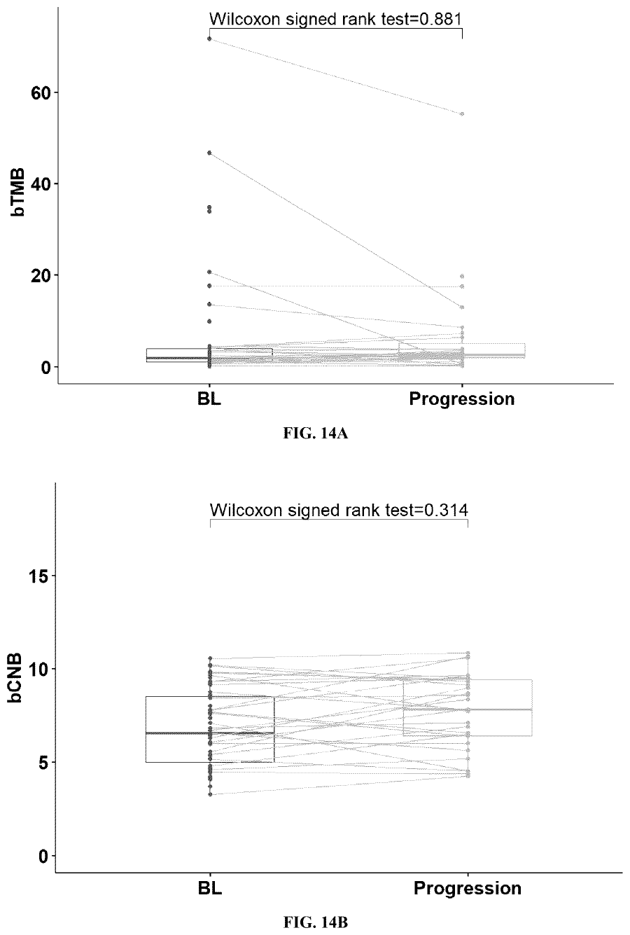

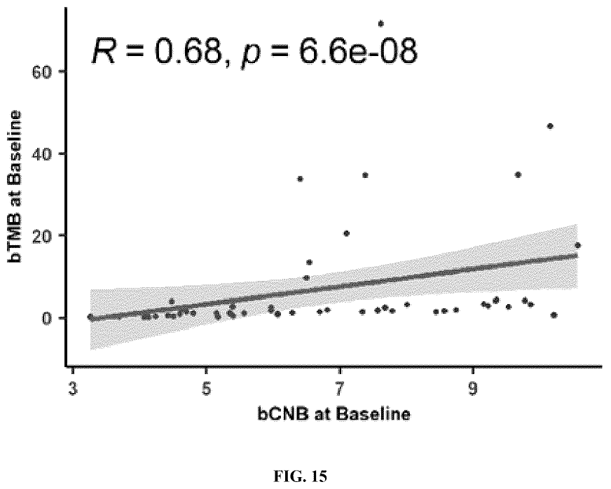

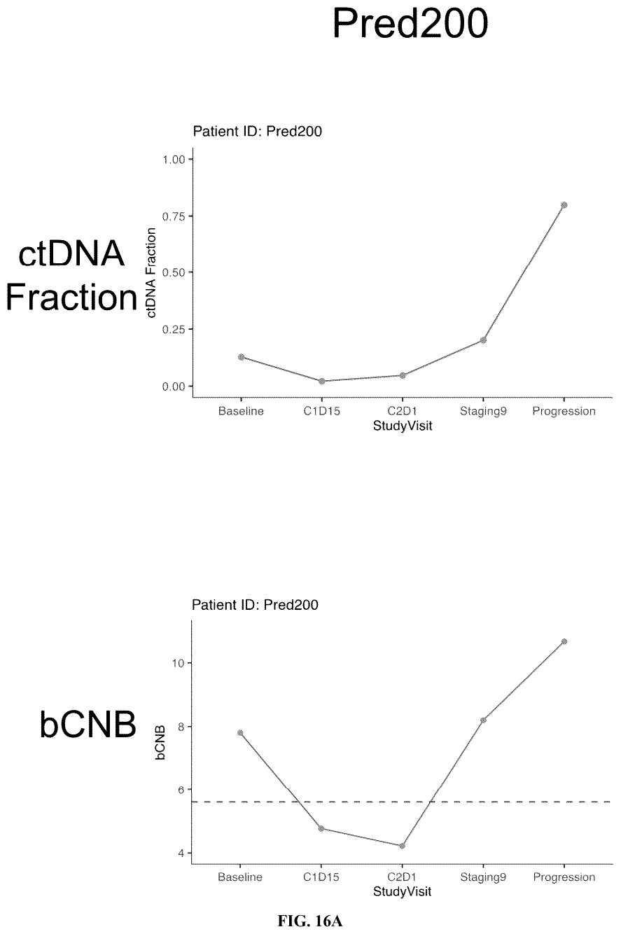

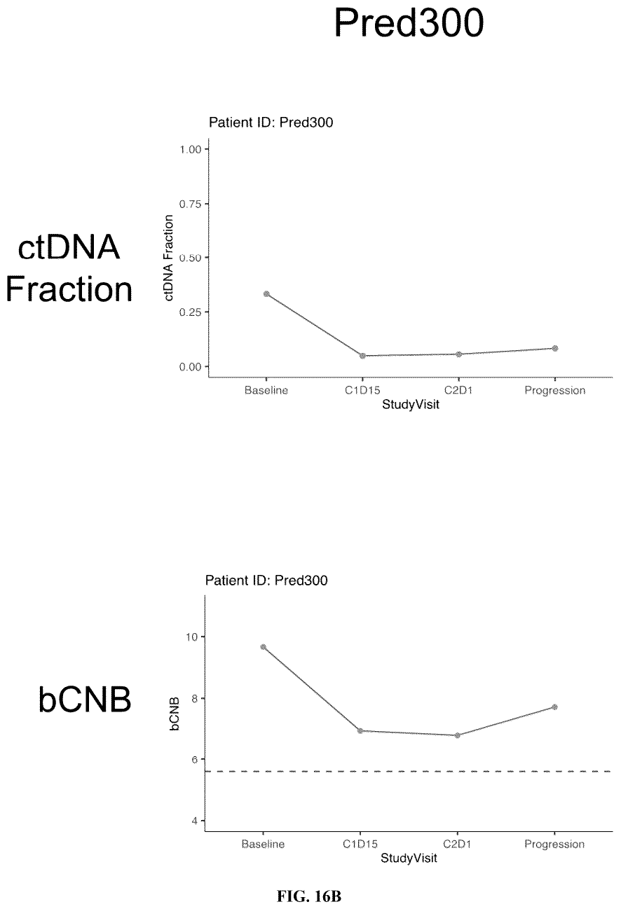

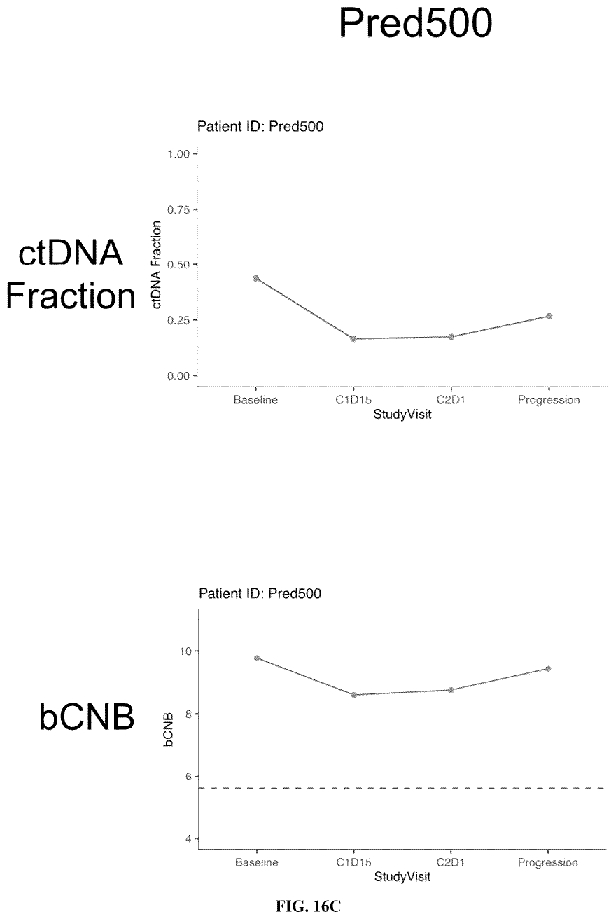

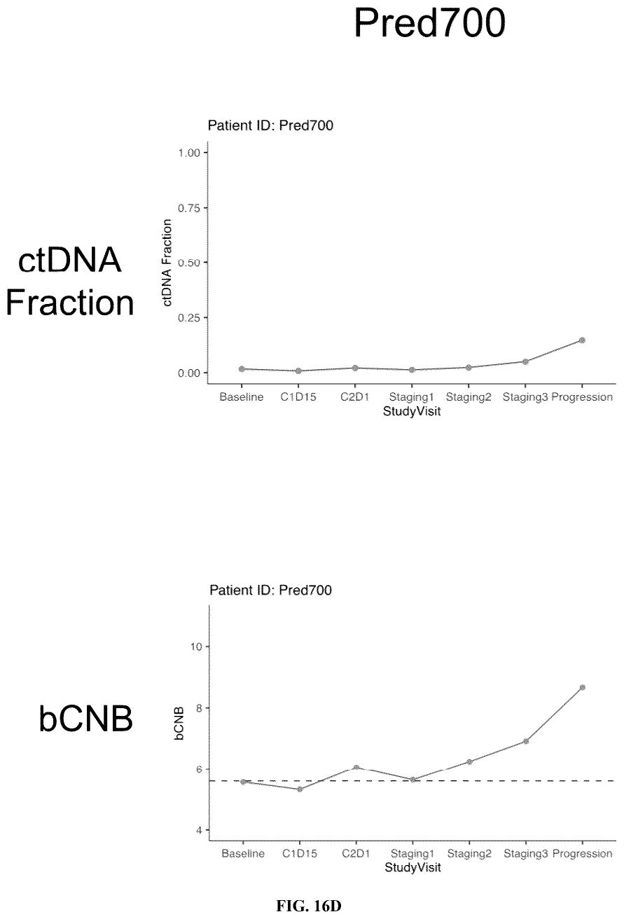

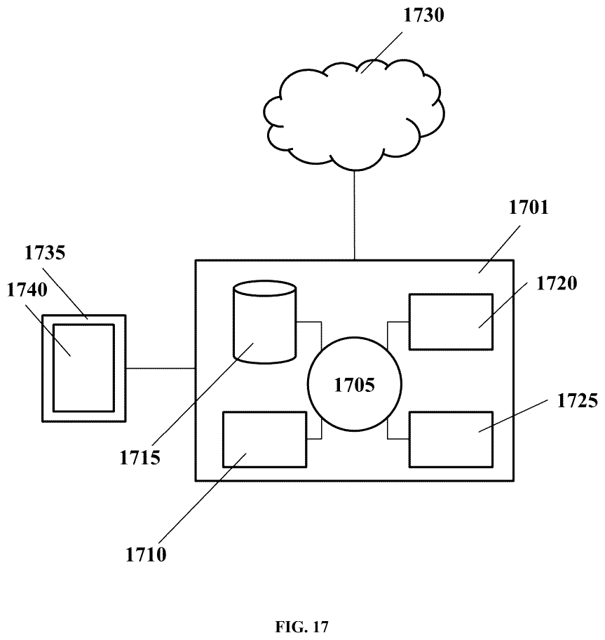

High bTMB scores were significantly associated with lack of clinical benefit (CB) defined as progressive disease (PD) within 6 months (FIG. 2B) and the presence of ESR1 mutations at baseline (Wilcoxon test) (FIG. 2C).

Clinical classification of endocrine resistance per ESMO 2020 guidelines did not predict bTMB, although there were a greater number of high bTMB patients in the endocrine resistant cohort (Kruskal-Wallis test) (FIG. 2D).

The association of high bTMB with significantly shorter PFS was observed using multiple cutoffs for bTMB including the median (FIG. 2E), third quartile (FIG. 2F), or bTMB scores of 10 mutations/megabase (FIG. 2G) (log rank test).

Within the endocrine resistant cohort, high bTMB scores were significantly associated with shorter PFS (log rank test) (FIG. 2H).

FIG. 3 shows high correlation between bTMB determined from WES and a 600-gene targeted sequencing panel. bTMB levels generated from 50 baseline samples using the PredicineWES™ and PredicineATLAS™ sequencing assays were highly correlated (Spearman's Rank Test).

FIGS. 4A-4B show that high bTMB at baseline is associated with dominant APOBEC mutational signatures and upregulation of specific oncogenic signaling pathways.

FIG. 4A shows that dominant APOBEC mutational signatures (SBS2 and SBS13) were enriched in patients with high (N=13) vs. low (N=37) bTMB scores (defined by upper quartile cutoff). Dominant SBS5 (unknown) and SBS6 (defective DNA mismatch repair signatures) were observed across patients with high and low bTMB scores.

FIG. 4B shows that the proportion of dominant APOBEC signatures was significantly greater in patients with high bTMB scores vs. low bTMB scores (Fisher's Exact Test).

FIG. 4C shows that the median bTMB score was significantly higher in patients with dominant APOBEC signatures vs. other signatures (Wilcoxon's Rank Sum Test).

FIGS. 5A-5B show that specific genomic alterations are enriched at disease progression.

FIG. 5A shows heatmaps comparing the most frequently altered genes detected at progression vs. baseline by PredicineWES+™ (N=28). Color coded bars below each heatmap indicate the level of bTMB, and the bar graph above the progression heatmap indicates PFS in months for each patient. Blue arrows denote patients with dominant APOBEC signatures.

FIG. 5B shows that enrichment at progression was observed for previously reported gene alterations implicated in resistance to endocrine and/or CDK4/6i therapy including a significantly higher frequency of RB1 alterations (P=0.04) (McNemar's test), and non-significant enrichment in AR, AURKA, CCND1, CDKNKA, ESR1, FGFR, MYC, PTEN and TP 53 alterations. In addition, non-significant enrichment of gene alterations not commonly linked to ET+CDK4/6i resistance such as BRCA2, CBL, CDH, KMT2D, MUC12 and PREX2 was observed at progression. Two of the enriched gene alterations (MUC12 and PREX2) were not covered by the targeted PredicineATLAS™ panel and were detected only through comprehensive WES.

FIGS. 6A-6C show that scores predict patient outcomes and increase before radiographic detection of clinical progression.

FIG. 6A shows that baseline bCNB scores were significantly higher in patients who experienced progression within 6 months (without clinical benefit) (Wilcoxon test).

FIG. 6B shows that high baseline bCNB scores were significantly associated with shorter PFS (log rank test).

FIG. 6C shows that serial analysis of bCNB during treatment revealed decreases at C1D15 and/or C2D1, followed by increases that preceded imaging detection of progressive disease in 12/18 (66.7%) of patients for whom staging blood samples were analyzed.

FIGS. 7A-7C show that high bTMB is associated with the presence of specific gene alterations. High baseline bTMB scores were significantly associated with higher frequencies of mutations in ARIDIA, DNAH10, and MUC16 (FIG. 7A), BSN, DSP, and PIK3CA (FIG. 7B), and CDH1, MUC6, and USH2A (FIG. 7C) (Wilcoxon test).

FIG. 8 shows receiver operating characteristic (ROC) analysis to determine optimal cutoff point of bTMB for clinical benefit. To gauge the predictive ability of baseline bTMB and to identify an optimal cutoff point for CB, we conducted ROC analysis on baseline bTMB with clinical benefit (CB). The ROC curve was plotted for each potential cutoff on the bTMB score, with estimated AUC(=0.76, 95% CI=0.574˜0.946) and the optimal cutoff point identified at 3.154 with the corresponding specificity (=0.775) and sensitivity (=0.7). The optimal cutoff point of 3.154 coincides with the upper 75% quartile (=3.8) of patients' bTMB, which was used to dichotomize patients throughout this study, unless otherwise stated.

FIG. 9 shows high correlation between bTMB determined from WES and a 152-gene targeted sequencing panel. bTMB levels generated from 50 baseline samples using the PredicineWES™ and PredicineCARE™ sequencing assays were highly correlated (Spearman's Rank Test).

FIGS. 10A-10B show that high cfDNA yield is associated with shorter progression-free survival (PFS).

FIG. 10A shows that high cfDNA yield in plasma samples collected from 51 patients at baseline was significantly associated with shorter PFS compared to patients with lower cfDNA yield, as defined by median and upper quartile cutoffs (log rank test).

FIG. 10B shows that high cfDNA yield in plasma samples collected from 22 patients with endocrine resistance was significantly associated with shorter PFS, as defined by median and upper quartile cutoffs (log rank test).

FIG. 11 shows that bTMB scores are not associated with sites of metastatic spread. bTMB scores at baseline are not significantly different across patients with vs. without bone and/or visceral disease (Kruskal-Wallis test).

FIGS. 12A-12B show that specific oncogenic signaling pathways are more frequently altered in patients with high bTMB and bCNB scores.

FIG. 12A shows that significantly higher frequencies of alterations were observed in high vs. low bTMB patients across breast cancer driver genes in the Cell Cycle, DNA Damage Repair (DDR), Hippo, Notch, PIK3 and Receptor Tyrosine Kinase (RTK-RAS, Hippo and PI3K oncogenic signaling pathways (Fisher's Exact Test).

FIG. 12B shows that significantly higher frequencies of alterations were also observed in high vs. low bCNB patients across breast cancer driver genes in the Cell Cycle, DDR, Hippo, Notch, RTK-RAS and TP53 oncogenic signaling pathways (Fisher's Exact Test).

FIG. 13 shows oncoprint showing landscape of genomic alterations across all patients at baseline. Frequencies of alterations across all 51 patients, including SNVs and CNVs across all patients are shown on the right. The bars on top of each column represent the total number of alterations detected in a given patient.

FIGS. 14A-14B show bTMB and bCNB at baseline vs. progression timepoints. No significant differences were observed in median bTMB or bCNB levels detected in baseline vs. progression samples (N=28) (Wilcoxon signed rank test).

FIG. 15 shows correlation of baseline bTMB and bCNB scores. bTMB and bCNB scores across 50 patients at baseline were highly correlated (Spearman's Rank Correlation Coefficient).

FIGS. 16A-16D show a comparison of ctDNA dynamics as measured by ctDNA fraction and bCNB during treatment and progression. Serial blood samples from 4 patients (Pred200 in FIG. 16A, Pred300 in FIG. 16B, Pred500 in FIG. 16C, and Pred700 in FIG. 16C) were analyzed by PredicineATLAS™ to derive ctDNA fraction values and by PredicineCNB™ to derive bCNB scores at each of 5 different timepoints (baseline, CID15, C2D1, staging 1-3, and progression). Similar dynamics are observed using both NGS metrics.

FIG. 17 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Provided herein are systems and methods for detection of the presence or absence of cancer in a subject. The systems and methods provided herein comprises assaying polynucleotides to identify biomarkers of cancers in a subject. The biomarkers may be processed in order to identify the presence or absence of cancer. The methods described herein may process multiple type of analytes in order to determine a presence or absence of cancer. The multiple types of analytes may comprise DNA or RNA, for example cfDNA or cfRNA. The multiple analytes may be cfDNA, germline DNA, and cfRNA. By analyzing a plurality of different analytes the methods may allow for improved detection or determination of a prognosis as compared to methods performed on fewer analytes or only one of many different analytes.

In an aspect, the present disclosure provides a method for detecting a presence or an absence of cancer in a subject, comprising: (a) assaying cell-free deoxyribonucleic acid (cfDNA) molecules and cell-free ribonucleic (cfRNA) molecules from a biological sample obtained or derived from the subject to detect a first set of biomarkers from the cfDNA molecules and a second set of biomarkers from the cfRNA molecules; and (b) computer processing the first set of biomarkers and the second set of biomarkers to detect the presence or the absence of the cancer in the subject.

The subject may be a suspected of a suffering from a cancer. The cancer may be specific or originating from an organ or other area of the subject. For example, the cancer may be breast cancer, lung cancer, prostate cancer, colorectal cancer, melanoma, bladder cancer, non-Hodgkin lymphoma, kidney cancer, endometrial cancer, leukemia, pancreatic cancer, thyroid cancer, and liver cancer, and any combination thereof. The cancer may be a hormone sensitive prostate cancer (HSPC), castrate-resistant prostate cancer (CRPC), metastatic prostate cancer, and a combination thereof. The cancer may comprise biomarkers that are specific to a particular cancer. The specific biomarkers may indicate a presence of a particular cancer. For example, biomarker may indicate that a castrate-resistant prostate cancer is present. The identification of the presence of a type of cancer may allow the determination of a treatment option or recommendation.

In some cases, the subject may be asymptomatic for cancer. For example, the cancer may not exhibit any symptoms and the subject may be unaware of the presence of cancer. The methods described herein may allow a cancer to be identified at an earlier stage than otherwise. The identification of the presence of the cancer at an earlier stage may allow a treatment option or recommendation to be determined at an earlier stage and may allow the subject to have an improved prognosis.

The biological sample may comprise nucleic acids. The biological sample be a cell-free deoxyribonucleic acid (cfDNA) sample or a cell-free ribonucleic acid (cfRNA) sample. The biological sample may comprise genomic DNA or germline DNA (gDNA). The nucleic acid may be a DNA (e.g. double-stranded DNA, single-stranded DNA, single-stranded DNA hairpins, cDNA, genomic DNA, germline DNA, circulating tumor DNA (ctDNA), cell-free DNA (cfDNA)), an RNA (e.g. cfRNA, mRNA, CRNA, miRNA, siRNA, miRNA, snoRNA, piRNA, tiRNA, snRNA), or a DNA/RNA hybrids. The biological sample may be a derived from or contain a biological fluid. For example, the biological sample may be a plasma sample, a serum sample, a buffy coat sample, a peripheral blood mononuclear cell (PBMC) sample, a red blood cell sample, a urine sample, a saliva sample, or other body fluid sample. The biological sample may comprise or be a pleural fluid sample, peritoneal fluid sample, amniotic fluid sample, cerebrospinal fluid sample, lymphatic fluid sample, sweat sample, tear sample, semen sample, or any combination of biological fluid. In some case, the samples may comprise RNA and DNA. For example, a sample may comprise cfDNA and cfRNA and the cfDNA and cfRNA may be analyzed by methods as described elsewhere herein.

The biological sample may be collected, obtained, or derived from the subject using a collection tube. The collection tube may be an ethylenediaminetetraacetic acid (EDTA) collection tube, a cell-free RNA collection tube, or a cell-free deoxyribonucleic acid (DNA) collection tube and CTC collection tubes, or other blood collection tube. The collection tube may comprise additional reagents for stabilizing the nucleic acid molecules or blood cells. The collection tube may allow the nucleic acid or blood cells to be stable such to minimize degradation of the biological sample prior to assaying. The additional reagents may comprise buffer salts or chelators.

The biological sample may be obtained or derived from a subject at a various times. The biological sample may be obtained or derived from a subject prior to the subject receiving a therapy for cancer. The biological sample may be obtained or derived from a subject during receiving a therapy for cancer. The biological sample may be obtained or derived from a subject after receiving a therapy for cancer. The biological sample may be collected over 1, 2, 3, 4, 5, 6, 7, 8, 9, 10, 11, 12, 15, 20, 30, 40, 50, 60, 70, 80, 90, 100, 200, 300, 400, 500, 600, 700, 800, 900, 1000 or time points. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more hour period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more day period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more week period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more month period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more year period.

In various aspects as described herein, a clinical intervention or a therapy may be identified at least in part based on the identification of the presences of cancer, or the presence of a parameter of cancer. The clinical intervention may be a plurality of clinical interventions. The clinical intervention may be selected from a plurality of clinical interventions. The clinical intervention may be a surgical resection, chemotherapy, radiotherapy, immunotherapy, adjuvant therapy, neoadjuvant therapy, androgen deprivation therapy, or a combination thereof. In some cases, the clinical interventions may be administered to the subject. After administration of the clinical intervention, a sample may be obtained or derived from the subject such to monitor the cancer or cancer parameters. As such, the methods and systems disclosed herein may be performed iteratively such that monitoring of a cancer can be performed. Additionally, by performing the methods or systems iteratively, therapies or clinical interventions may be updated based on the results of the methods. The monitoring of the cancer may include an assessment as well as a difference in assessment from a previously generated assessment. The difference in an assessment of cancer in the subject among a plurality of time points (or samples) may be indicative of one or more clinical indications such as a diagnosis of the cancer, a prognosis of the cancer, or an efficacy or non-efficacy of a course of treatment for treating the cancer of the subject. The prognosis may comprise expected progression-free survival (PFS), overall survival (OS), or other metrics relating the severity or survivability of a cancer

The biological samples may be subjected to additional reactions or conditions prior to assaying. For example, the biological sample may be subjected to conditions that are sufficient to isolate, enrich, or extract nucleic acids, such cfDNA molecules or cfRNA molecules.

The methods disclosed herein may comprise conducting one or more enrichment reactions on one or more nucleic acid molecules in a sample. The enrichment reactions may comprise contacting a sample with one or more beads or bead sets. The enrichment reactions may comprise one or more hybridization reactions. For example, the enrichment reactions may comprise contacting a sample with one or more capture probes or bait molecules that hybridize to a nucleic acid molecule of the biological sample. The enrichment reaction may comprise differential amplification of a set of nucleic acid molecules. The enrichment reaction may enrich for a plurality of genetic loci or sequences corresponding to genetic loci. For example, the enrichment reaction may enrich for sequences corresponding to genes from Table 1 or Table 2. The enrichment reactions may comprise the use of primers or probes that may complementarity to sequences (or sequences upstream or downstream) of a sequence that is to be enriched. For example, a capture probe may comprise sequence complementarity to a set of genomic loci and allow the enrichment of the genomic loci. The enrichments reactions may comprise a plurality of probes or primers. A plurality of probes may comprise 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 different probes.

The methods disclosed herein may comprise conducting one or more isolation or purification reactions on one or more nucleic acid molecules in a sample. The isolation or purification reactions may comprise contacting a sample with one or more beads or bead sets. The isolation or purification reaction may comprise one or more hybridization reactions, enrichment reactions, amplification reactions, sequencing reactions, or a combination thereof. The isolation or purification reaction may comprise the use of one or more separators. The one or more separators may comprise a magnetic separator. The isolation or purification reaction may comprise separating bead bound nucleic acid molecules from bead free nucleic acid molecules. The isolation or purification reaction may comprise separating capture probe hybridized nucleic acid molecules from capture probe free nucleic acid molecules. The isolation reactions may comprises removing or separating a group of nucleic acid molecules from another group of nucleic acids.

The methods disclosed herein may comprise conduction extraction reactions on one or more nucleic acids in a biological sample. The extraction reactions may lyse cells or disrupt nucleic acid interactions with the cell such that the nucleic acids may be isolated, purified, enriched or subjected to other reactions.

The methods disclosed herein may comprise amplification or extension reactions. The amplification reactions may comprise polymerase chain reaction. The amplification reaction may comprise PCR-based amplifications, non-PCR based amplifications, or a combination thereof. The one or more PCR-based amplifications may comprise PCR, qPCR, nested PCR, linear amplification, or a combination thereof. The one or more non-PCR based amplifications may comprise multiple displacement amplification (MDA), transcription-mediated amplification (TMA), nucleic acid sequence-based amplification (NASBA), strand displacement amplification (SDA), real-time SDA, rolling circle amplification, circle-to-circle amplification or a combination thereof. The amplification reactions may comprise an isothermal amplification.

The method disclosed herein may comprise a barcoding reaction. A barcoding reaction may comprise the additional of a barcode or tag to the nucleic acid. The barcode may be a molecular barcode or a sample barcode. For example, a barcode nucleic acid may comprise a barcode sequence which may be a degenerate n-mer. The sequence may be randomly generated or generated such to synthesize a specific barcode sequence. The barcode nucleic acid may be added to a sample such to label the nucleic acid molecules in the sample. The barcodes may be specific to a sample. For example, a plurality of barcode nucleic acids may be added to a sample in which the barcode sequence is the same. Upon barcoding of the nucleic acids, those originating from a same sample may have a same barcode sequence, and may allow a nucleic acid to be identified as belonging to a particular or given sample. A molecular barcode may also be used such that each molecule (or a plurality of molecules) in a same volume have a different molecular barcode. This barcode may be subjected to amplification such that all amplicons derived from a molecule have the same barcode. In this way, molecules originating from a same molecule may be identified. The sequences reads may be processed based on the barcode sequences. For example, the processing may reduce errors or allow a molecule to be tracked. Barcode sequences may be appended or otherwise added or incorporated into a sequence by various reactions, for example an amplification, extension, or ligation reaction, and may be performed enzymatically using a nucleic acid polymerase or ligase. The ligation may be an overhang or blunt end ligation and the barcodes may comprise complementarity to nucleic acids to be barcoded. This complementarity may be a sequence derived from the sample from the subject or may be constant sequence generated via a reaction performed on the nucleic acids in the sample.

In some cases, the biological sample may comprise multiple components. For example, the biological sample may be a whole blood sample. The biological sample may be subjected to reactions such to separate or fractionate a biological sample. For example, a whole blood sample may be a fractionated and cell free nucleic acids may be obtained. The whole blood sample may be fractionated using centrifugation such that blood cells may be separated from the plasma (which may contain cell free nucleic acid). A sample may be subjected to multiple rounds of separation or fractionation.

In various aspects described throughout the disclosure, the nucleic acids may be subjected to sequencing reactions. The sequencing the reactions may be used on DNA, RNA or other nucleic acid molecules. Example of a sequencing reaction that may be used include capillary sequencing, next generation sequencing, Sanger sequencing, sequencing by synthesis, single molecule nanopore sequencing, sequencing by ligation, sequencing by hybridization, sequencing by nanopore current restriction, or a combination thereof. Sequencing by synthesis may comprise reversible terminator sequencing, processive single molecule sequencing, sequential nucleotide flow sequencing, or a combination thereof. Sequential nucleotide flow sequencing may comprise pyrosequencing, pH-mediated sequencing, semiconductor sequencing or a combination thereof. The sequencing reactions may comprise whole genome sequencing, whole exome sequencing, low-pass whole genome sequencing, targeted sequencing, methylation-aware sequencing, enzymatic methylation sequencing, bisulfite methylation sequencing. The sequencing reaction may be a transcriptome sequencing, mRNA-seq, totalRNA-seq, smallRNA-seq, exosome sequencing, or combinations thereof. Combinations of sequencing reactions may be used in the methods described elsewhere herein. For example, a sample may be subjected to whole genome sequencing and whole transcriptome sequencing. As the samples may comprise multiple types of nucleic acids (e.g. RNA and DNA), sequencing reactions specific to DNA or RNA may be used such to obtain sequence reads relating to the nucleic acid type.

The sequencing of nucleic acids may generate sequencing read data. The sequencing reads may be processed such to generate data of improved quality. The sequencing reads may be generated with a quality score. The quality score may indicate an accuracy of a sequence read or a level or signal above a nose threshold for a given base call. The quality scores may be used for filtering sequencing reads. For example, sequencing reads may be removed that do not meet a particular quality score threshold. The sequencing reads may be processed such to generate a consensus sequence or consensus base call. A given nucleic acid (or nucleic acid fragment) may be sequenced and errors in the sequence may be generated due to reactions prior or during sequencing. For example, amplification or PCR may generate error in amplicons such that the sequences are not identical to a parent sequence. Using sample barcodes or molecular barcodes, error correction may be performed. Error correction may include identifying sequence reads that do not corroborate with other sequences from a same sample or same original parent molecules. The use of barcodes may allow the identification or a same parent or sample. Additionally, the sequence reads may be processed by performing single strand consensus calling or double stranded consensus call, thereby reducing or suppressing error.

The methods as disclosed herein may comprise determining allele frequency or other cancer related metric. The methods may comprise a mutant allele frequency of a set of somatic mutation among a set of biomarkers. The mutant allele frequency may be used to determine a circulating tumor DNA (ctDNA) fraction of a cancer of a subject. A plasma tumor mutational burden (pTMB) of a cancer of the subject may be determined based at least in part on the set of mutant allele frequencies. Detection of microsatellite instability may also be used to determine the presence or absence of a cancer or cancer metric. Methylation states may be determined using methods described herein and may be used to identify a presence of a cancer or cancer parameter.

In various aspects, sets of biomarkers are processed and data corresponding to the biomarkers are generated. The sets of biomarkers may comprise quantitative measures from a set of cancer-associated genomic loci. The cancer-associated genomic loci may correspond to a set of genes. The cancer associated genomic loci may comprise one or more genes selected from Table 1. In some case, a set of cancer associated genomic loci comprises 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 members selected from the group consisting of genes listed in Table 1. The cancer associated genomic loci may comprise one or more genes selected from Table 2. In some case, a set of cancer associated genomic loci comprises 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 members selected from the group consisting of genes listed in Table 2.

TABLE 1
List of genes

| | | | | | | | |
|---|---|---|---|---|---|---|---|
| ABRAXAS1 | AKT1 | ALK | APC | AR | ARAF | ARIDIA | ATM |
| ATR | BAP1 | BARD1 | BRAF | BRCA1 | BRCA2 | BRIP1 | CCND1 |
| CCNE1 | CD274 | CDH1 | CDK12 | CDK4 | CDK6 | CDKN2A | CHEK1 |
| CHEK2 | CTNNB1 | DDR2 | DNAJB1 | EGFR | EPCAM | ERBB2 | ERBB3 |
| ERCC1 | ERCC2 | ERCC4 | ESR1 | EZH2 | FANCA | FANCC | FANCD2 |
| FANC1 | FANCL | FANCM | FBXW7 | FGFR1 | FGFR2 | FGFR3 | GNA11 |
| GNAQ | GNAS | HDAC2 | HRAS | IDH1 | IDH2 | JAK2 | JAK3 |
| KIT | KRAS | MAP2K1 | MAP2K2 | MAPK1 | MDM2 | MET | MLH1 |
| MPL | MRE11 | MSH2 | MSH6 | MTOR | MYC | MYCN | MYD88 |
| NBN | NF1 | NFE2L2 | NPM1 | NRAS | NTRK1 | NTRK3 | PALB2 |
| PDCD1LG2 | PDGFRA | PIK3CA | PMS2 | POLD1 | POLE | PPM1D | PPP2R1A |
| PPP2R2A | PRKACA | PRKD1 | PTEN | PTPN11 | RAD50 | RAD51 | RAD51B |
| RAD51C | RAD51D | RAD54L | RAF1 | RB1 | RECQL | RET | RNF43 |
| ROS1 | RPA1 | SMAD4 | SMO | SPOP | STK11 | TERT | TMPRSS2 |
| TP53 | TP53BP1 | TSC1 | TSC2 | VHL | XRCC2 | XRCC3 | XRCC4 |

TABLE 2
List of genes in PredicineCARE panel
Gene List
PredicinePLUS DNA + RNA Panel
SNV and ind [symbol] (15 [symbol] k [symbol] y cancer [symbol] genes)

| | | | | | | | | | | |
|---|---|---|---|---|---|---|---|---|---|---|
| ABL1 | BRCA1* | CREBBP | EZH1 | H3F3A | MAPZK1 | NF1* | PIK3CA* | RAD51C* | SMARCA4 | VEGFA* |
| AKT1 | BRCA2* | CRKL* | EZH2 | H3F3B | MAP2K2 | NF2* | PIX3CB | RAD51D | SMARCB1* | VEGFB |
| ALK | BTK | CSF1R | FANCA | HNF1A | MAPK1 | NFE2L2 | PIK3CD | RAF1 | SMO* | VHL |
| APC* | CALR | CTNNB1 | FBXW7 | HRAS | MDM2* | NOTCH1 | PIK3R1* | RB1* | SPOP | WT1 |
| AR* | CBL | CXCR4 | FGFR1* | IDH1 | MED12 | NPM1 | PM52* | RET | SR5F2 | ZNF148 |
| ARAF | CCND1* | DOR2 | FGFR2* | IDH2 | MET* | NRAS | POLD1 | RHEB | STAG2 | |
| AR1D1A* | CCND2 | DICER1 | FGFR3* | IKZF1 | MLH1* | NTRK1* | POLE | RHOA | STAT3 | |
| AR1D2 | CCND3 | DNMT3A | FGFR4* | JAK2 | MPL | NTRK2* | PPP2R1A | RIT1 | STK11 | |
| ASXL1 | CD274* | EGFR* | FLT3 | JAK3 | MSH2* | NTRK3* | PRKACA | RNF43 | TERT | |
| ATM* | CD798 | EPCAM | FOXL2* | KCNJS | MSH6* | PALB2* | PRKD1 | ROS1 | TET2 | |
| ATR | CDH1* | ERBB2* | GATA2 | KDMGA* | MTOR | PAX5* | PTCH1* | RUNX1 | TGFBR2 | |
| ATRX | CDK4* | ERBB3 | GATA3 | KDR | MUTYH | PBRM1* | PTEN* | SERPINB3* | TP53* | |
| BZM | CDK6* | ERBB4 | GNA11 | KIT* | MYC* | PDCD1LG2* | PTPN11* | SERPINB4* | TSC1 | |
| BAP1* | CDKN2A* | ERRFI1* | GNAQ | KMT2C | MYCN* | PDGFRA | RAC1 | SF3B1 | TSC2 | |
| BRAF | CHEK2* | ESR1 | GNAS | KRAS* | MYD88 | PDGFRB | RAD51B* | SMAD4 | U2AF1 | |

Chromotherapy response related genes (9 genes)

| | | | | | | | | |
|---|---|---|---|---|---|---|---|---|
| CYP2C19 | CYP2D6 | CYP3A4 | DPYD | GSTP1 | MTHFR | UGT1A1 | XPC | XRCC1 |

Copy number alteration detection only (15 genes)

| | | | | | | | | | | |
|---|---|---|---|---|---|---|---|---|---|---|
| AKT2 | AREG | AURKA | BCL2 | CCNE1 | CDKN2B | EREG | FGF19 | FGF3 | FGF4 | HGF |
| IGF1R | IGF2 | TOP2A | PPP2R2A | | | | | | | |

Detection of [symbol] by Breakpoints: 12 genes ( [symbol] genes present above)

| | | | | | | | | | | |
|---|---|---|---|---|---|---|---|---|---|---|
| ALK | FGFR1 | FGFR3 | MET | PDGFRA | RET | CD74 | EGFR | FGFR2 | MET | NTRK1 |
| PDGFRB | ROS1 | | | | | | | | | |

*indicated genes covered by full coding regions [symbol]
indicates data missing or illegible when filed

The sets of biomarkers may correspond to genetic aberration of a genetic locus. The genetic aberration may a tumor associated alteration. The genetic aberration may be a copy number alterations (CNAs), copy number losses (CNLs), single nucleotide variants (SNVs), insertions or deletions (indels), and rearrangements. The set of biomarkers may be identified in a variety of nucleic acid types. For example, the tumor associated alteration may be identified in cfDNA or cfRNA. The tumor associated alteration may comprise changes in allelic expression, or gene expression. Methods and systems disclosed herein may allow for gene expression profiling and identification of changes to the expression levels of gene.

In various aspects, the methods may comprise identifying the presence of a cancer or a cancer parameter. The methods may comprises determining a probability or a likelihood of the presence of cancer or a cancer parameter. For example, instead of a binary output indicating a presence or absence, an output may be generated that indicates a probability that subject has cancer. This probability may be determined based on algorithms as described elsewhere herein. Similarly, a probability or likely of response to a particular treatment or a probability of relapse may be outputted.

The increased cfRNA transcriptional expression of drug resistance-related gene alterations or splicing variants may serve as predictive biomarker, identifying the response or resistance to therapy. Specifically, in the case of prostate cancer, the increased cfRNA transcriptional expression of drug resistance-related AR mutations such as W742C/L and F877L or splicing variants such as AR-V7 or AR-V9, may serves as predictive biomarker, identifying the response or resistance to anti-androgen therapy.

Compared to the use of cfDNA, blood ctRNA-based variant detection (including fusion) can be used to be more effectively to identify known and novel variants especially fusions in cancer. For instance, blood cfRNA based detection of TMPRSS2-ERG provides higher detection sensitivity in prostate cancer.

The increased ratio of blood-based cancer variants versus urine-based cancer variants could serve as a prognostic biomarker in GU cancers, indicating the disease aggressiveness and guide clinical treatment decision making. Specifically, in the case of muscle-invasive bladder cancer (MIBC), the increased level of blood-based cancer variants versus urine-based cancer variants could serve as a prognostic biomarker in patients with MIBC and provide evidence for clinical decision making. These cancer variants may include ctDNA, cfRNA, microRNA, methylation, among others.

Together with cfDNA based variant detection through genomics and epigenomics, cfRNA and/or microRNA can also be used either alone or in combination with genomic and epigenomic biomarkers for minimal residual disease (MRD) detection, therapy monitoring and early cancer detection.

In various aspects, the sets of biomarkers are processed using an algorithm. The algorithm may be a trained algorithm. The trained algorithms may use the sets of biomarkers as an input and generate an output regarding the presence or absence of a cancer. The output may be specific to a type of cancer or subtype of cancer. For example, the output may indicate the presence of a castrate-resistant prostate cancer.

The trained algorithm may be trained on multiple samples. For example, the trained algorithm may be trained using at least 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 300, 400, 500, 600,700, 800, 900, 1000, 1100, 1200, 1300, 1400, 1500, 1600, 1700, 1800, 1900, 2000, 3000, 4000, 5000, 6000, 7000, 8000, 9000, 10000, or more independent training samples. The trained algorithm may be trained using no more 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 300, 400, 500, 600,700, 800, 900, 1000, 1100, 1200, 1300, 1400, 1500, 1600, 1700, 1800, 1900, 2000, 3000, 4000, 5000, 6000, 7000, 8000, 9000, 10000, or less, independent training samples. The training samples may be associated with a presence or an absence of the cancer. The training samples may be associated with a relapse of cancer. The training samples may be associated with cancer that is resistant to a particular drug or treatment. An individual training sample may be positive for a particular cancer. An individual training sample may be negative for a particular cancer. By using training samples, the trained algorithm may be able to detect a cancer, determine a probability of recurrence or relapse of a cancer, or determine if a cancer comprises a set of biomarkers may be resistant to a treatment. The training sample may be associated with additional clinical health data of a subject. For example, additional clinical health data may comprise the gender, weight, height, or levels of metabolites or antibodies in a subjects. Additional clinical health data may comprise indication of other diseases, disorders, or diseases conditions.

The trained algorithms may be trained using multiple sets of training samples. The sets may comprise training samples as described elsewhere herein. For example, the training may be performed using a first set of independent training samples associated with a presence of the cancer and a second set of independent training samples associated with an absence of the cancer. Similarly, a first set may be associated with relapse and a second sample may be associated with the absence of relapse.

The trained algorithm may also process additional clinical health data of the subject. For example, additional clinical health data may comprise the gender, weight, height, or levels of metabolites or antibodies in a subjects. Additional clinical health data may comprise indication of other diseases, disorders, or diseases conditions that the subject may suffer from. By using the additional clinical health data, in conjunction with the biomarkers, the trained algorithm may output a presence or absences of cancer, probability of relapse, or resistance to drug treatment, that may be different from the output of an algorithm that does not process additional clinical health.

The trained algorithm may be an unsupervised machine learning algorithm. For example, the unsupervised machine learning algorithm may utilize cluster analysis to identify attributes of interest. The trained algorithm may be a supervised machine learning algorithm. For example, the algorithm may be inputted with training data such to generate an expected or desired output. The supervised learning algorithm may comprise a deep learning algorithm, a support vector machine (SVM), a neural network, or a Random Forest. Via the machine learning algorithm, the trained algorithm may be able to identify relationships of biomarkers to a particular cancer prognosis or diagnosis. Without the trained algorithm, it may otherwise difficult to identify relationships of the biomarkers to accurately identify the presence of a cancer or other parameters associated with the cancer.

In various aspects, the systems and methods may comprise a accuracy, sensitivity, or specificity of detection of the cancer or a parameter of the cancer. For example, the methods or systems may comprise detecting the presence or the absence of cancer (or the presence of a parameter of the cancer, such as recurrence, relapse, or drug resistance) in the subject at an accuracy of at least about 60%, at least about 70%, at least about 75%, at least about 80%, at least about 85%, at least about 90%, at least about 95%, at least about 98%, or at least about 99%. The methods or systems may comprise detecting the presence or the absence of cancer (or the presence of a parameter of the cancer, such as recurrence, relapse, or drug resistance) in the subject at a sensitivity of at least about 60%, at least about 70%, at least about 75%, at least about 80%, at least about 85%, at least about 90%, at least about 95%, at least about 98%, or at least about 99%. The methods or systems may comprise detecting the presence or the absence of cancer (or the presence of a parameter of the cancer, such as recurrence, relapse, or drug resistance) in the subject at a specificity of at least about 60%, at least about 70%, at least about 75%, at least about 80%, at least about 85%, at least about 90%, at least about 95%, at least about 98%, or at least about 99%. The methods or systems may comprise detecting the presence or the absence of cancer (or the presence of a parameter of the cancer, such as recurrence, relapse, or drug resistance) in the subject at a positive predictive value of at least about 60%, at least about 70%, at least about 75%, at least about 80%, at least about 85%, at least about 90%, at least about 95%, at least about 98%, or at least about 99%. The methods or systems may comprise detecting the presence or the absence of cancer (or the presence of a parameter of the cancer, such as recurrence, relapse, or drug resistance) in the subject at a negative predictive value of at least about 60%, at least about 70%, at least about 75%, at least about 80%, at least about 85%, at least about 90%, at least about 95%, at least about 98%, or at least about 99%.

### Computer Systems

The present disclosure provides computer systems that are programmed to implement methods of the disclosure. FIG. 17 shows a computer system 1701 that is programmed or otherwise configured to perform analysis or steps of the methods, for example, determining a tumor mutational burden, determining a copy number burden, or running an algorithm. The computer system 1701 can regulate various aspects of methods and systems of the present disclosure, such as, for example, perform an algorithm, input training data, analyze sets of biomarker, or output a result for the user as to the tumor mutational burden or the copy number burden. The computer system 1701 can be an electronic device of a user or a computer system that is remotely located with respect to the electronic device. The electronic device can be a mobile electronic device.

The computer system 1701 includes a central processing unit (CPU, also “processor” and “computer processor” herein) 1705, which can be a single core or multi core processor, or a plurality of processors for parallel processing. The computer system 1701 also includes memory or memory location 1710 (e.g., random-access memory, read-only memory, flash memory), electronic storage unit 1715 (e.g., hard disk), communication interface 1720 (e.g., network adapter) for communicating with one or more other systems, and peripheral devices 1725, such as cache, other memory, data storage and/or electronic display adapters. The memory 1710, storage unit 1715, interface 1720 and peripheral devices 1725 are in communication with the CPU 1705 through a communication bus (solid lines), such as a motherboard. The storage unit 1715 can be a data storage unit (or data repository) for storing data. The computer system 1701 can be operatively coupled to a computer network (“network”) 1730 with the aid of the communication interface 1720. The network 1730 can be the Internet, an internet and/or extranet, or an intranet and/or extranet that is in communication with the Internet. The network 1730 in some cases is a telecommunication and/or data network. The network 1730 can include one or more computer servers, which can enable distributed computing, such as cloud computing. The network 1730, in some cases with the aid of the computer system 1701, can implement a peer-to-peer network, which may enable devices coupled to the computer system 1701 to behave as a client or a server.

The CPU 1705 can execute a sequence of machine-readable instructions, which can be embodied in a program or software. The instructions may be stored in a memory location, such as the memory 1710. The instructions can be directed to the CPU 1705, which can subsequently program or otherwise configure the CPU 1705 to implement methods of the present disclosure. Examples of operations performed by the CPU 1705 can include fetch, decode, execute, and writeback.

The CPU 1705 can be part of a circuit, such as an integrated circuit. One or more other components of the system 1701 can be included in the circuit. In some cases, the circuit is an application specific integrated circuit (ASIC).

The storage unit 1715 can store files, such as drivers, libraries and saved programs. The storage unit 1715 can store user data, e.g., user preferences and user programs. The computer system 1701 in some cases can include one or more additional data storage units that are external to the computer system 1701, such as located on a remote server that is in communication with the computer system 1701 through an intranet or the Internet.

The computer system 1701 can communicate with one or more remote computer systems through the network 1730. For instance, the computer system 1701 can communicate with a remote computer system of a user (e.g., a medical professional or patient). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1701 via the network 1730.

Methods as described herein can be implemented by way of machine (e.g., computer processor) executable code stored on an electronic storage location of the computer system 1701, such as, for example, on the memory 1710 or electronic storage unit 1715. The machine executable or machine readable code can be provided in the form of software. During use, the code can be executed by the processor 1705. In some cases, the code can be retrieved from the storage unit 1715 and stored on the memory 1710 for ready access by the processor 1705. In some situations, the electronic storage unit 1715 can be precluded, and machine-executable instructions are stored on memory 1710.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1701, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1701 can include or be in communication with an electronic display 1735 that comprises a user interface (UI) 1740 for providing, for example, an input of biomarkers or sequencing data, or an visual output relating to a detection, diagnosis, or prognosis. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1705. The algorithm can, for example, determine a tumor mutational burden or a copy number burden.

### EXAMPLES

### Example 1: Analysis of Genome-Wide ctDNA for Evaluating bTMB and bCNB

CDK4/6 inhibition (CDK4/6i) in combination with endocrine therapy (ET) improves survival for patients with hormone receptor-positive (HR+)/HER2-negative (HER2−) metastatic breast cancer (MBC). However, clinical biomarkers to identify patients who will not respond are lacking. Using methods and systems of the present disclosure, genome-wide circulating tumor DNA (ctDNA) analysis was performed to identify features associated with resistance to ET and CDK4/6i.

ctDNA was isolated from 216 plasma samples collected from 51 patients with HR+/HER2− MBC at baseline and during treatment on a phase II trial of palbociclib combined with letrozole or fulvestrant (NCT3007979). Boosted whole exome sequencing (WES) was performed at baseline and clinical progression to profile genomic alterations, evaluate mutational signatures, and derive blood tumor mutational burden (bTMB). Low-pass whole-genome sequencing was performed at baseline, serial timepoints on therapy, and clinical progression to assess blood copy number burden (bCNB).

Results were obtained, including that high bTMB and bCNB were associated with lack of clinical benefit and significantly shorter progression-free survival (PFS) compared to patients with low bTMB or low bCNB (all P<0.05). Dominant APOBEC signatures were detected at baseline exclusively in cases with high bTMB (5/13, 38.5%) vs. low bTMB (0/37, 0%) (P=0.0006). Previously reported and novel alterations were detected at baseline and progression in association with treatment resistance. Alterations in ESR1 were enriched in samples with high bTMB (P=0.0005). There was a high correlation between bTMB determined by WES and bTMB determined using a 600-gene panel (R=0.98). During serial monitoring, an increase in bCNB preceded radiographic progression in 12/18 (66.7%) patients.

Results showed that genomic complexity demonstrated by high bTMB and bCNB was associated with lack of response and poor outcome for patients treated with ET and CDK4/6i. This subset of HR+/HER2− patients requires exploration of novel treatment strategies including immunotherapy-based combinations. Non-invasive monitoring in blood was performed to identify the emergence of resistance alterations and early evidence of progression before imaging.

The combination of endocrine therapy (ET) and cyclin-dependent kinase 4/6 inhibition (CDK4/6i) has emerged as the standard-of-care, first-line treatment for patients with hormone-receptor positive (HR+)/HER2− negative metastatic breast cancer (MBC). This treatment indication is based on the significant improvement in survival outcomes and extended chemotherapy-free interval across all clinical and pathological subgroups [1-5]. Therefore, outside of clinical trials or impending organ failure, patients in the United States and Europe are offered CDK4/6i and ET as first-line treatment. Despite this advancement in care for patients with HR+/HER2− negative MBC, a subset of patients rapidly progress, and biomarkers to predict efficacy and resistance are lacking.

Analysis of circulating tumor DNA (ctDNA) using next-generation sequencing (NGS) enables the non-invasive assessment of genomic alterations during tumor progression and may be used to identify biomarkers for predicting and monitoring response to treatment [6-10]. In 2019, the Food and Drug Administration (FDA) approved a ctDNA-based companion diagnostic test for the detection of PIK3CA mutations to select patients for treatment with alpelisib, leading to increased utilization of ctDNA tests in clinical practice [11, 12]. Both tissue and blood-based NGS profiling have identified individual alterations associated with resistance in patients treated with ET with CDK4/6i, including alterations in CCNE1, FGFR1, FAT1, PTEN, and RB1 [13-18]. However, to date, no clinical, pathological, or genomic signatures have been identified as predictive at baseline to define a subset of patients who benefit from alternative treatment strategies. Therefore, a comprehensive NGS-based liquid biopsy approach encompassing assessment of ctDNA mutation and copy number burden was developed for identifying prognostic and predictive biomarkers in patients with HR+/HER2-negative MBC and track response to ET and CDK4/6i treatment. To accomplish this, we utilized a combination assay that provides targeted coverage of 600 cancer genes in addition to whole exome sequencing (WES) to enable comprehensive genomic profiling, evaluation of mutational signatures, and derivation of bTMB at baseline and progression timepoints. In addition, we implemented low-pass whole genome sequencing (LP-WGS) to derive a novel measure of genome-wide copy number variation (CNV).

Tumor mutational burden (TMB), as used herein, generally refers to a measure of the number of mutations per megabase of sequenced DNA, which may be measured using, for example, WES [19]. The rationale for developing TMB as a clinical biomarker, initially derived from tissue, may be based on observations that tumor types with high tissue TMB (tTMB) (e.g. non-small cell lung cancer (NSCLC) in smokers, melanoma associated with ultraviolet radiation, and mismatch repair deficient tumors) respond well to immune checkpoint inhibitor (ICI) therapy [20-23]. tTMB may show promise as a potential surrogate biomarker for neoantigen load to predict response to ICI monotherapy and as a non-overlapping biomarker in conjunction with PD-L1 expression on tumor or immune cells [24]. Blood tumor mutational burden (bTMB) may be explored as a non-invasive method of TMB determination in NSCLC, given the difficulty of obtaining adequate tissue for sequencing in some cases, and studies performed with NGS targeted cancer gene panels may show that NSCLC patients with high bTMB preferentially responded to ICI over chemotherapy [25-27]. However, the application of WES to measure TMB in blood samples may face various technical challenges [28, 29]. Relative to other malignancies, evaluation of TMB in breast cancer has been less extensive, with many studies instead assessing (TMB. The evaluation of tTMB may show that, while patients with breast cancer have a relatively low median tTMB, tTMB is higher in metastatic versus primary tissue. Importantly, early data may show that a subset of breast cancer patients with high TMB benefit from PD-1 inhibitors with or without anti-CTLA-4 [30-32]. In addition, parallel assessment of mutational signatures in hypermutated malignancies may show the presence of APOBEC (alipoprotein B mRNA-editing enzyme catalytic polypeptide-like) mutational signatures in high tTMB patients, which may be associated with response to ICI [30, 33-35].

Blood copy number burden (bCNB), derived from the PredicineCNB™ assay, is a comprehensive measure of CNV via LP-WGS, including amplifications and deletions across the entire genome. While current strategies for blood-based treatment response monitoring may primarily track individual ctDNA mutations or changes in allele frequency to evaluate tumor response to systemic therapy, the integration of copy number changes and whole-genome methylation can provide an early signal of response for patients treated with a variety of systemic therapies prior to standard-of-care imaging [36-40]. Given that LP-WGS is less expensive compared to other NGS methods and therefore more feasible for serial testing from a cost perspective, the technique may offer clinical applications for monitoring dynamic changes in CNV during the course of treatment. However, studies evaluating this technique in patients with MBC treated with CDK4/6i are limited.

Using systems and methods of the present disclosure, two novel, genome-wide ctDNA assays were used that combine sequencing breadth and depth to profile patients with HR+/HER2-negative MBC who are receiving combined ET and CDK4/6i treatment in a prospective phase II interventional clinical trial. Resistance biomarkers were determined to assess which patients may be suitable candidates for novel treatment strategies and to explore the potential for serial ctDNA monitoring to predict early disease progression. Our comprehensive approach identified bTMB and bCNB levels that predicted poor patient outcomes, identified APOBEC signatures exclusively in hypermutated patients, defined an expanded list of candidate alterations that may mediate resistance at baseline and clinical progression, and demonstrated the use of bCNB for prediction and monitoring of early disease progression.

A patient cohort was obtained as follows. Patient ctDNA samples were retrospectively analyzed from a prospective, single-arm, phase II study (NCT03007979) that was conducted at the Washington University School of Medicine (St. Louis, MO) and the University of Nebraska Medical Center (Omaha, NE). HR+/HER2-negative MBC patients treated with 0-1 lines of prior systemic therapy without prior use of CDK4/6i were enrolled. Patients received palbociclib 125 mg daily, on a continuous 5-days-on and 2-days-off weekly schedule in combination with letrozole or fulvestrant (per physician's choice) with goserelin administration for premenopausal patients. Each treatment cycle was 28 days. Research blood samples were collected in Streck tubes at baseline, cycle 1 day 15 (C1D15), cycle 2 day 1 (C2D1), and cycle 4 day 1 (C4D1), and then on D1 of every 3 cycles (with tumor imaging) until disease progression. Fifty-four patients were enrolled to the study, of which 51 patients were evaluable for response and included in this analysis. At data cutoff, 29 patients were taken off study due to disease progression, and therefore samples were available for 29 patients at disease progression. For these patients, plasma samples collected at the timepoints immediately prior to clinical progression were also included in this analysis. The results of the primary endpoint (rate of grade 3 or 4 neutropenia), and clinical response were obtained [41]. The study was approved by the institutional review board at each site, and informed written consent was obtained from all patients to allow correlative research on their blood samples

ctDNA analysis was performed as follows. Patient samples were analyzed using two comprehensive NGS platforms, PredicineWES+™ and PredicineCNB™ (Predicine, Inc., Hayward, CA), to generate genomic profiles, perform mutational signature and pathway analyses, and derive measures of bTMB and bCNB. Briefly, cell-free DNA (cfDNA) extracted from patient plasma samples and germline DNA extracted from peripheral blood mononuclear cells (PBMCs) were processed and subjected to library construction. The resulting DNA libraries were sequenced by PredicineCNB™ LP-WGS at a depth of coverage of 5× or further enriched by hybrid capture, and sequencing was performed with PredicineWES+™, a combination assay designed to sequence the entire exome with sequencing depth at 2,500× (1% level of detection (LOD)) along with boosted sequencing of 600 cancer genes covered by the PredicineATLAS™ targeted panel with sequencing depth at 20,000× (0.25% LOD) (Table 3). PredicineWES+™ sequencing data were used to generate the landscape of genomic alterations including single-nucleotide variants (SNVs), insertions and deletions (indels), CNVs, and gene fusions, to derive bTMB scores reporting the total number of somatic mutations detected per megabase of DNA and analyze mutational signatures and oncogenic signaling pathway involvement. bTMB scores were also derived from sequencing data generated by analysis using a targeted 600-gene PredicineATLAS™ panel and a targeted 152-gene PredicineCARE™ panel (Table 4) in order to compare bTMB values generated by PredicineWES+™. PredicineCNB™ sequencing data were evaluated to generate bCNB scores representing a comprehensive genome-wide measure of CNV, including amplifications and deletions across the entire genome.

[symbol]

TABLE 3
List of 600 genes targeted by the PredicineATLAS ™ panel

| | | | | | | |
|---|---|---|---|---|---|---|
| ABL1 | ABL2 | ABRAXAS1 | ACVR1 | ACVR1B | ACVR2A | ADARB2 |
| ALOX5 | AMER1 | ANKRD11 | APC | APEX1 | AR | ARAF |
| ARIDS8 | ASXL1 | ASXL2 | ATM | ATR | ATRX | AURKA |
| BAP1 | BARD1 | BCL30 | BCL2 | BCL211 | BCL2L11 | BCL2L2 |
| BLM | BMPR1A | BRAF | BRCA1 | BRCA2 | BRD2 | BRD3 |
| CARD11 | CARM1 | CASP8 | CBFB | CBL | CCND1 | CCND2 |
| CO70 | CD74 | CO79A | CB798 | CDC42 | CDC7 | COC73 |
| CDKN38 | CDKN2A | CDKN28 | CDKN2C | CEBPA | CENPA | CHEK1 |
| CSF1R | CSF3R | CTCF | CTLA4 | CTNNA1 | CTNN81 | CUL3 |
| CYP2D6 | CVP3A4 | CY5CTR2 | DAXX | DDIT3 | DDR3 | DDR2 |
| DNMT3A | DNMT38 | DOT1L | DPY0 | DUSP4 | DYRK2 | E2F3 |
| ELOC | EML4 | EMSY | EP300 | EPAS1 | EPCAM | EPHA3 |
| ERBB4 | ERCC1 | ERCC2 | ERCC3 | ERCC4 | ERCCS | ERCC6 |
| ETV1 | ETV4 | ETV5 | ETV6 | EWSR1 | EXO1 | EZH1 |
| FANCA | FANC8 | FANCC | FANCD2 | FANCE | FANCF | FANCG |
| FEN1 | FGF10 | FGF12 | FGF14 | FGF19 | FGF23 | FGF3 |
| FH | FLCN | FLI1 | FLT2 | FLT3 | FLT4 | FOXA1 |
| FZD1 | F2D10 | FZD2 | F2D3 | FZD4 | F2DS | FZD6 |
| GATA6 | GEN1 | GID4 | GLI1 | GNA11 | GNA13 | GNAQ |
| GSTP1 | H3F3A | H3F38 | H3F3C | HDAC1 | HDAC2 | HELQ |
| HLA-8 | HNF1A | HOXB13 | HRAS | HSD383 | HSP90AA1 | ID3 |
| IGF1 | IGF1R | IGF2 | IGF2R | IKBKE | IK2F1 | IL10 |
| IRF2 | IRF4 | IRS2 | JAK1 | JAK2 | JAK3 | JUN |
| KDR | KEAP3 | KIT | KLF4 | KLHL6 | KMT2A | KMT2C |
| LIG4 | LMO1 | LYN | MAD2L2 | MALT1 | MAP2K1 | MAP2K2 |
| MAPK3 | MAPKAP1 | MAX | MCL1 | MDC1 | MDM2 | MDM4 |
| MLH1 | MLH3 | MLST8 | MPL | MRAS | MRE11 | MSH2 |
| MYB | MYC | MYCL | MYCN | MYD88 | MYOD1 | NBN |
| NKX2-1 | NKX3-1 | NOTCH1 | NOTCH2 | NOTCH3 | NOTCH4 | NPM1 |
| NT5C2 | NTHL1 | NTRK1 | NTRK2 | NTRK3 | NUMB | NUP93 |
| PARP2 | PARP3 | PAXS | PBRM1 | PDCD1 | PDCD38G2 | PDGFRA |
| PIAS4 | PIK3C28 | PIK3C2G | PIK3C3 | PIK3CA | PIK3CB | PIK3CD |
| PLEKHS1 | PML | PMS1 | PMS2 | POLD1 | POLE | PDLH |
| PPP2R2A | PPP3CA | PPP4R2 | PPP6C | PRDM1 | PRDM14 | PREX2 |
| PICH1 | PTEN | PTPN11 | PTPRD | PTPRY | QK1 | RAB35 |
| RADS18 | RAD51C | RADS1D | RADS2 | RADS4L | RAF1 | RARA |
| RET | REV3L | RGS1 | RHE8 | RHOA | RHOB | RICTOR |
| RPS6KA3 | RPTOR | RRAGC | RRAS | RRAS2 | RSPO1 | RSPO2 |
| SDHAF2 | SDHB | SDHC | SDHD | SEM1 | SERPIN83 | SERPINB4 |
| SHOC2 | SHQ1 | SLC34A2 | SLFN11 | SLX4 | SMAD2 | SMAD3 |
| SOCS1 | SOCS3 | SOS1 | SOX10 | SOX17 | SOX2 | SOX9 |
| STAG2 | STAT3 | STAT4 | S? | STK11 | STK19 | SUFU |
| TERT | TET1 | TET2 | TGFBR1 | TGFBR2 | TMEM127 | TMPRSS2 |
| TPS3 | TP53BP1 | TP63 | TP73 | TRAF2 | TRAF3 | TRAF2 |
| UBE2T | UGT1A1 | VEGFA | VEGF8 | VHL | VTCN1 | WEE1 |
| XPC | XPO1 | XRCC1 | XRCC2 | XRCC3 | XRCC4 | XRCC5 |
| ZNRF3 | ZRSR2 | | | | | |

| | | | | | |
|---|---|---|---|---|---|
| AKT1 | AKT1S1 | AKT2 | AKT3 | ALK | ALOX128 |
| AREG | ARFRP1 | ARHGAP35 | ARID1A | ARID18 | ARID2 |
| AURKB | AXIN1 | AXIN2 | AXL | B2M | BABAM1 |
| BCL6 | BCOR | BCGRL1 | BCR | BIRC3 | BIRC5 |
| BRD4 | BRIP1 | BTG1 | BTG2 | BTK | CALR |
| CCND3 | CCNE1 | CCNQ | CD22 | CD274 | CD276 |
| CDH1 | CDK12 | CDK4 | CDK6 | CDK8 | CDKN1A |
| CHER2 | CIC | CNOT3 | CREBBP | CRKL | CRLF2 |
| CUL4A | CUX1 | CXCR4 | CYLD | CYP17A1 | CYP2C19 |
| DEPDC5 | DEPTOR | DICER1 | DIS3 | DLL4 | DNAJ81 |
| EED | EGFR | EIF1AX | EIF4A2 | EIF4E | ELF3 |
| EPHA5 | EPHA7 | EPHB1 | EPHB4 | ERBB2 | ER883 |
| ERCC8 | EREG | ERF | ERG | ERRFI1 | ESR1 |
| E2H2 | EZR | FAAP100 | FAAP20 | FAAP24 | FAM45C |
| FANC1 | FANCL | FANCM | FAS | FAT1 | FBKW7 |
| FGF4 | FGF6 | FGFR1 | FGFR2 | FGFR3 | FGFR4 |
| FOXL2 | FOXO1 | FOXP1 | FRS2 | FUBP1 | FUS |
| F2D7 | FZD8 | F2D9 | GATA1 | GATA2 | GATA3 |
| GRA5 | GPS2 | GREM1 | GRIN2A | GRM3 | GSK35 |
| HES1 | HEY1 | HEYL | HGF | HIST3H3 | HLA-A |
| IDH1 | IDH2 | IDO1 | IFNG | IFNGR1 | IFNGR2 |
| IL2RG | IL78 | INHA | INHBA | INPP48 | IRF1 |
| KAT6A | KCNJS | KDM1A | KDM5A | KDMSC | KDM6A |
| XMT2O | KNSTRN | KRAS | LATS1 | LATS2 | LIG1 |
| MAP2K4 | MAP3K1 | MAP3K13 | MAP3K14 | MAP4K3 | MAPK1 |
| MED12 | MEF28 | MEN1 | MERTK | MET | MITF |
| MSH3 | MSH6 | MST1R | MTHFR | MTOR | MUTYH |
| NEGR1 | NF1 | NF2 | NFE2L2 | NFKBIA | NHEI1 |
| NPRL2 | NPRL3 | NRAS | NSD1 | NSD2 | NSD3 |
| NUTM1 | PZRY8 | PAK3 | PALB2 | PARG | PARP1 |
| PDGFRB | PDK1 | PDPK1 | PGR | PHF6 | PHOX28 |
| PIK3CG | PIK3R1 | PIK3R2 | PIK3R3 | PIM1 | PIN1 |
| POLQ | POU2F2 | PPARG | PPM1D | PPP2CA | PPP2R1A |
| PRKACA | PRKAR1A | PRKCI | PRKD1 | PRKDC | PRKN |
| RAC1 | RAC2 | RAD1B | RAD21 | RAD50 | RADS1 |
| RASA1 | RB1 | RBM10 | RECQL | RECQL4 | REL |
| RIT1 | RNF43 | ROBO1 | ROBO2 | ROS1 | RPA1 |
| RSPO4 | RUNX1 | RXRA | RYBP | SDC4 | SDHA |
| SESN2 | SETO2 | SF3B1 | SGK1 | SH283 | SH2D1A |
| SMAD4 | SMARCA2 | SMARCA4 | SMARC83 | SMARCD1 | SMO |
| SPEN | SPOP | SRC | SRSF2 | SRY | SS18 |
| SUZ32 | SYK | TBC1O7 | TBX3 | TEK | TERC |
| TNFAIP3 | TNFRSF14 | TNFRSF1A | TNK2 | TOP1 | TOP2A |
| T5C1 | TSC2 | T5HR | TSH22 | TYRO3 | U2AF1 |
| WISP3 | WRN | WT1 | XBP1 | XIAP | XPA |
| XRCC6 | YAP1 | ZFHX3 | ZNF148 | ZNF217 | ZNF703 |

* Full exonic coverage for all genes except ones used for fusion detection. Fusions genes underlined.

TABLE 4
List of 152 genes targeted by the PredicineCARE ™ panel

| | | | | | | | | | |
|---|---|---|---|---|---|---|---|---|---|
| [symbol] | [symbol] [symbol] | [symbol] [symbol] | [symbol] [symbol] | [symbol] [symbol] | [symbol] [symbol] | [symbol] [symbol] | [symbol] | [symbol] [symbol] | [symbol] [symbol] |
| [symbol] [symbol] | [symbol] | [symbol] | [symbol] | [symbol] [symbol] | [symbol] [symbol] | [symbol] [symbol] | [symbol] | [symbol] | [symbol] [symbol] |
| [symbol] [symbol] | [symbol] [symbol] | [symbol] [symbol] | [symbol] [symbol] | [symbol] [symbol] | [symbol] [symbol] | [symbol] | [symbol] [symbol] | [symbol] [symbol] | [symbol] [symbol] |
| [symbol] [symbol] | [symbol] [symbol] | [symbol] | [symbol] | [symbol] | [symbol] | [symbol] | [symbol] | [symbol] | [symbol] |
| [symbol] | [symbol] | [symbol] [symbol] | [symbol] [symbol] | [symbol] | [symbol] [symbol] | [symbol] [symbol] | [symbol] | [symbol] | [symbol] |
| [symbol] | [symbol] | [symbol] | [symbol] | [symbol] | [symbol] [symbol] | [symbol] | [symbol] | [symbol] [symbol] | [symbol] [symbol] |
| [symbol] [symbol] | [symbol] [symbol] | [symbol] | [symbol] | [symbol] | [symbol] [symbol] | [symbol] | [symbol] [symbol] | [symbol] | [symbol] |
| [symbol] | [symbol] | [symbol] | [symbol] | [symbol] | [symbol] | [symbol] [symbol] | [symbol] | [symbol] | [symbol] [symbol] |
| [symbol] | [symbol] | [symbol] [symbol] | [symbol] | [symbol] [symbol] | [symbol] | [symbol] | [symbol] [symbol] | [symbol] [symbol] | [symbol] |
| [symbol] | [symbol] | [symbol] | [symbol] | [symbol] | [symbol] | [symbol] [symbol] | [symbol] [symbol] | [symbol] | [symbol] |
| [symbol] [symbol] | [symbol] | [symbol] | [symbol] | [symbol] | [symbol] [symbol] | [symbol] [symbol] | [symbol] | [symbol] [symbol] | [symbol] [symbol] |
| [symbol] [symbol] | [symbol] [symbol] | [symbol] | [symbol] | [symbol] | [symbol] | [symbol] | [symbol] [symbol] | [symbol] | [symbol] [symbol] |
| [symbol] | [symbol] [symbol] | [symbol] | [symbol] | [symbol] | [symbol] | [symbol] | [symbol] | [symbol] | [symbol] [symbol] |
| [symbol] [symbol] | [symbol] [symbol] | [symbol] | [symbol] | [symbol] | [symbol] | [symbol] [symbol] | [symbol] | [symbol] | [symbol] [symbol] |
| [symbol] | [symbol] | [symbol] | [symbol] | [symbol] [symbol] | [symbol] [symbol] | [symbol] | [symbol] | [symbol] | [symbol] |

| | | | | | | | |
|---|---|---|---|---|---|---|---|
| [symbol] | [symbol] | □ SNVs + Indels [symbol] | [symbol] CNVs | [symbol] Fusions | [symbol] Fusions + CNVs | [symbol] | [symbol] |

[symbol]

indicates data missing or illegible when filed

Statistical analysis was performed as follows. Statistical associations among individual alterations, bTMB, and bCNB with clinical benefit rate (CBR), defined as the percentage of patients with a complete response, partial response, or stable disease lasting at least 24 weeks by RECIST (version 1.1), were analyzed using Wilcoxon and Kruskal-Wallis tests. Frequencies of alterations across patient subgroups were compared using the Fisher's Exact test. Comparison of the frequencies of alterations across patient subgroups at baseline and clinical progression timepoints was performed using McNemar's Test. The degree of association between variables was evaluated with Spearman's Rank correlation coefficient. The Kaplan-Meier (K-M) method was applied to estimate empirical survival probabilities with K-M curves used to illustrate survival, and the log rank test was utilized to compare differences in survival. Hazard ratios and 95% confidence intervals were estimated from univariate Cox proportional hazards regression analysis. Different cutoffs were applied to bTMB for analysis of association with PFS, including the unbiased cutoffs of median and third quartile, while optimal cutoffs were further explored based on Harrell's C-index in a Cox model setting for PFS and receiving operating characteristic (ROC) analysis for clinical benefit. Changes in bCNB were assessed at serial timepoints and compared to concurrent assessment of clinical progression based on RECIST 1.1.

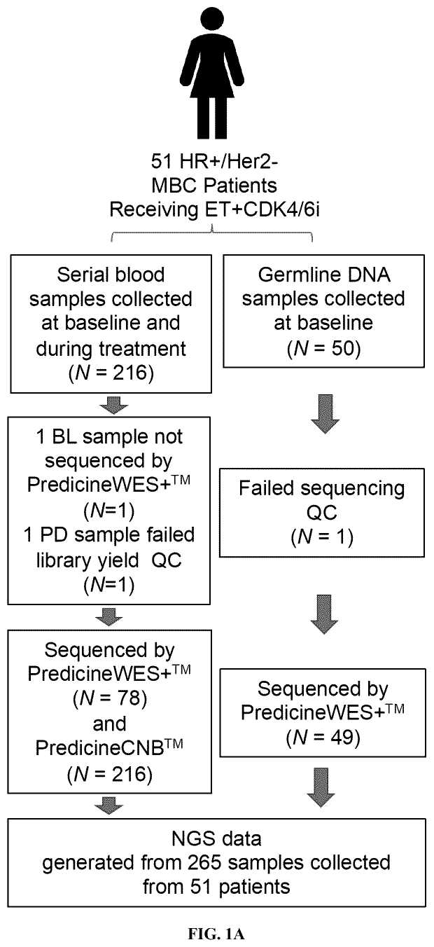

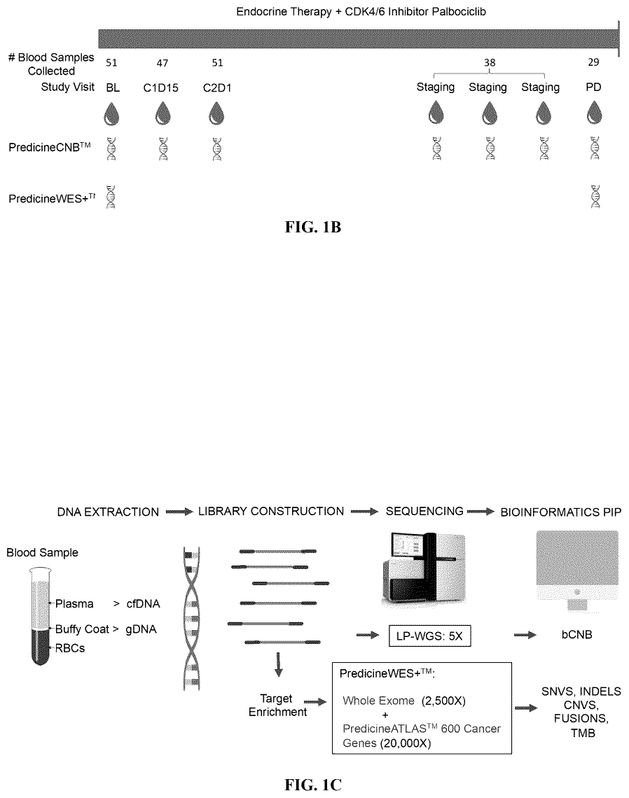

Serial ctDNA samples were analyzed from a prospective clinical trial as follows. ctDNA testing was performed retrospectively on samples collected from a prospective clinical trial of palbociclib in combination with ET (letrozole or fulvestrant) [41]. 265 samples from 51 evaluable patients with HR+/HER2-negative MBC were analyzed using Predicine liquid biopsy NGS platforms (FIG. 1A). At the time of data censoring, there were 29 patients who had progressed on study at a median follow-up time of 16.4 months (range 1.4-50.9). Only two (2/265) samples failed sequencing quality control, and greater than 99% of ctDNA samples were successfully sequenced. PredicineWES+™ analysis was performed on 78 plasma samples collected at baseline (n=50) and clinical progression (n=28), combining WES at 2,500× (1% LOD) and boosted sequencing (20,000×, LOD 0.25%) of 600 genes. PredicineCNB™ was performed at a sequencing depth of 5× for 216 plasma samples including all available baseline (n=51), C1D15 (n=47), C2D1 (n=51), staging evaluations prior to progression (n=38), and clinical progression (n=29) timepoints (FIGS. 1B-1C).

Clinical and pathological characteristics of patients included in the study are summarized in Table 5. The vast majority of patients were postmenopausal (84.3%) and received letrozole (72.6%), with the remaining patients receiving fulvestrant (27.5%). A total of 17 patients were de novo metastatic, 22 patients were classified as endocrine resistant, and 12 patients were endocrine sensitive based on ESMO 2020 criteria [42].

TABLE 5
Patient demographics

| | | | Clinical | Clinical | |
|---|---|---|---|---|---|
| | | | Benefit = | Benefit = | P |
| Variable | | All | No | Yes | value |
| Age (median, | | 62(53~67) | 64(57~66) | 59(53~68) | 0.65 |
| IQR) | | | | | |
| Race | | | | | 1 |
| | Black or | 9(17.65) | 2(20) | 7(17.07) | |
| | African | | | | |
| | American | | | | |
| | White | 42(82.35) | 8(80) | 34(82.93) | |
| ECOG | | | | | 0.17 |
| | 0 | 28(54.9) | 6(60) | 22(53.66) | |
| | 1 | 19(37.25) | 2(20) | 17(41.46) | |
| | 2 | 4(7.84) | 2(20) | 2(4.88) | |
| Menopausal | | | | | 1 |
| Status | | | | | |
| | Postmenopausal | 43(84.31) | 9(90) | 34(82.93) | |
| | Premenopausal | 8(15.69) | 1(10) | 7(17.07) | |
| Tumor Grade | | | | | 0.53 |
| | 1 | 8(17.78) | 2(22.22) | 6(16.67) | |
| | 2 | 30(66.67) | 7(77.78) | 23(63.89) | |
| | 3 | 7(15.56) | 0(0) | 7(19.44) | |
| Endocrine | | | | | 0.11 |
| Therapy | | | | | |
| | Fulvestrant | 14(27.45) | 5(50) | 9(21.95) | |
| | Letrozole | 37(72.55) | 5(50) | 32(78.05) | |
| Line of Therapy | | | | | 0.81 |
| | 1st Line | 47(92.15) | 9(90) | 38(92.7) | |
| | 2nd Line | 4(7.84) | 1(10) | 3(7.32) | |
| Treatment Setting | | | | | 0.45 |
| | De novo | 17(33.33) | 3(30) | 14(34.15) | |
| | Metastatic | | | | |
| | Endocrine | 22(43.14) | 6(60) | 16(39.02) | |
| | Resistant | | | | |
| | Endocrine | 12(23.53) | 1(10) | 11(26.83) | |
| | Sensitive | | | | |
| Bone Metastasis at | | | | | 0.66 |
| Baseline | | | | | |
| | N | 10(19.61) | 1(10) | 9(21.95) | |
| | Y | 41(80.39) | 9(90) | 32(78.05) | |
| Visceral | | | | | 0.08 |
| Metastasis at | | | | | |
| Baseline | | | | | |
| | N | 25(49.02) | 2(20) | 23(56.1) | |
| | Y | 26(50.98) | 8(80) | 18(43.9) | |
| Best Treatment | | | | | P < |
| Response | | | | | 10E−5 |
| (RECIST) | | | | | |
| | CR | 3(5.88) | 0(0) | 3(7.32) | |
| | PR | 15(29.41) | 0(0) | 15(36.59) | |
| | SD | 27(52.94) | 4(40) | 23(56.1) | |
| | PD | 6(11.76) | 6(60) | 0(0) | |

Further, high baseline bTMB was demonstrated to be associated with worse clinical outcomes. bTMB was evaluable for 50 patients at baseline (FIG. 2A). The median bTMB was 1.85 mutations per megabase pair (MBp) [interquartile range (IQR) 1.01-3.86, range 0.1-71.7]. Patients without clinical benefit who experienced disease progression within 6 months (N=10) had a significantly higher bTMB compared to patients with clinical benefit (N=40) (median 8.90 mutations/MBp [IQR 2.30-31.2] versus median 1.63 [IQR 0.69-2.83], Wilcoxon,P=0.012) (FIG. 2B). Patients with an ESR1 mutation at baseline (N=8) also had significantly higher baseline bTMB (Wilcoxon, P=5.0×10⁻⁴) (FIG. 2C), and similar associations were detected for baseline mutations in the ARID1A, BSN, CDH1, DNAH3, DSP, MUC6, MUC16, PIK3CA, and USH2A genes (FIGS. 7A-7C). When comparing patients based on the clinical classifications of de novo, endocrine resistant, or endocrine sensitive MBC, no significant difference in baseline bTMB was observed (FIG. 2D). However, the majority of patients with high bTMB at baseline were present in the endocrine resistant cohort. ROC analysis was performed to determine the optimal cutpoint for bTMB in our dataset as 3.2 mutations/MBp with AUC 0.76 (FIG. 8), which aligns closely to the 3.8 bTMB value that dichotomizes patients above and below the third quartile. Higher bTMB at baseline was significantly associated with worse PFS (median 13.8 months vs. 32.1 months) based on the median of the sample (Hazard ratio (HR) 2.62 [confidence interval (CI) [1.21-5.66], P=0.011 (FIG. 2E). A similarly significant difference between high and low bTMB was observed based on the third quartile of the sample (median 6.5 months vs. 32.1 months) (HR 4.87 [CI 2.19-10.81], P=2.27×10⁻⁵) (FIG. 2F) and at a cutoff of 10 mutations/MBp (median 3.8 months vs. 22.3 months) (HR 7.15 [CI 2.82-18.13], P=1.94×10⁻⁶) (FIG. 2G). When assessing survival in the endocrine resistant cohort, a significant difference was observed with high baseline bTMB significantly associated with worse PFS (HR 4.76 [CI 1.52-14.97], P=0.004) (FIG. 2H). In summary, high baseline bTMB was determined to be significantly associated with lack of clinical benefit and shorter PFS, as measured using multiple pre-determined and experimentally determined cutoff points.

Further, we demonstrated that bTMB scores generated from targeted sequencing panels and WES are highly correlated. bTMB levels generated from 50 baseline samples using the PredicineWES™ assay were compared with values obtained using the targeted 600-gene PredicineATLAS™ and the 152-gene PredicineCARE™ sequencing assays. bTMB values obtained by PredicineWES+™ were highly correlated with levels derived from PredicineATLAS™ (R=0.98) (FIG. 3) and PredicineCARE™ (R=0.93) (FIG. 9) (Spearman's Rank Test). Together, these comparisons demonstrate that accurate bTMB scores can also be generated from shorter NGS gene panels.

Further, we demonstrated that high cfDNA yield was associated with significantly shorter PFS based on the median (HR 2.36 [CI 1.12-4.98], P=0.021) and third quartile (HR 2.96 [CI 1.34-6.54], P=0.006) cutoffs of the samples (FIG. 10). In the endocrine resistant cohort, high cfDNA yield was also associated with significantly shorter PFS based on the median (HR 3.45 [CI 1.18-10.14], P=0.017), and third quartile (HR 4.35 [CI 1.29-14.63], P=0.01) cutoffs. Endocrine sensitivity versus endocrine resistance and site of disease at clinical presentation (e.g. bone or visceral) did not predict CBR or PFS for patients treated with palbociclib in combination with ET (Table 5). Similarly, there was no significant association between sites of metastatic spread and bTMB at baseline (FIG. 11). In summary, high baseline cfDNA levels were determined to be associated with poor clinical outcomes but not with commonly assessed clinicopathological features.

Further, we demonstrated that dominant APOBEC mutational signatures are present exclusively in high bTMB patients. Off-target activity of the APOBEC family of mutator enzymes can generate somatic mutations across the genome leading to distinct mutational signatures that have been associated with the development and progression of multiple cancers [22, 43-44]. To assess the contribution of these mutational signatures to the genomic landscape of high vs. low bTMB patients in this cohort, sequencing data obtained by PredicineWES™ from 50 patients at baseline were evaluated for single base substitution (SBS) patterns, which were compared against the 94 curated reference SBS mutational signatures available in the COSMIC database. Dominant APOBEC signatures were identified exclusively in the high bTMB patients, whereas the other signatures were observed across high and low bTMB groups (FIG. 4A). Dominant APOBEC signatures were detected in 5/13 (38.5%) patients with high bTMB vs. 0/37 (0%) patients with low bTMB (P=0.0006, Fisher's Exact Test) (FIG. 4B). The median bTMB score was significantly higher in patients with dominant APOBEC signatures (34.8 MBp) vs. other signatures (1.7 MBp) (P=0.00048, Wilcoxon's Rank Sum Test) (FIG. 4C). Collectively, these findings show the determination of a subset of hypermutated HR+/HER2-negative patients with MBC.

Further, we demonstrated that specific oncogenic signaling pathways are more frequently altered in high bTMB and high bCNB patients To compare the relative proportion of alterations within key oncogenic signaling pathways in high vs. low bTMB and bCNB patients, we compared the frequencies of alterations identified across breast cancer driver genes present in 12 pathways [34-35, 45]. Significantly higher frequencies of alterations (including SNVs and CNVs) were observed in high vs. low bTMB patients across breast cancer driver genes in the Cell Cycle (P=0.04), DNA Damage Repair (DDR) (P=0.02), Hippo (P=0.009), NOTCH (P=0.003), PI3K (P=2.9×10⁻⁰⁵) and Receptor Tyrosine Kinase (RTK)-RAS (P=0.005) oncogenic signaling pathways (Fisher's Exact Test) (FIG. 12). Significantly higher frequencies of alterations were also observed in high vs. low bCNB patients in the Cell Cycle (P=0.009), DDR (P=0.001), Hippo (P=0.04), Notch (P=0.04), RTK-RAS (P=0.007) and TP53 (P=0.002) pathways (Fisher's Exact Test) (FIG. 12). These findings show that relevant signaling pathways that are implicated in driving ET and CDK4/6i resistance are preferentially activated in hypermutated, genomically complex breast cancer patients in this cohort.

Further, we demonstrated that comprehensive profiling extends detection of clinically relevant ctDNA alterations at baseline and detects enrichment of novel ctDNA alterations at progression. The PredicineWES+™ assay was performed on 50/51 samples collected at baseline and 28/29 samples collected at progression. One of 51 baseline samples was sequenced using the PredicineATLAS™ assay instead of the PredicineWES+™ assay, and one of the progression samples failed library yield quality control. The most frequently observed alterations across all 51 patients at baseline were PIK3CA (45%), TP53 (31%), and ESR1 (20%) (FIG. 13). Baseline alterations (SNVs and CNVs) in 17 genes were significantly associated with worse PFS, including AURKA, AKT3, ATM, BRCA2, CCND1, CCNE2, DDR2, DSP, ESR1, MYC, MUC16, PIK3CA, PLCGI, RB1, RUNXITI, USH2A and ZFHX3 (Table 6). Five of these genes (CCNE2, DSP, MUC16, PLCGI and USH2A) are not targeted by the PredicineATLAS™ panel and may not have been implicated in CDK4/6i and ET resistance, thereby representing novel resistance alterations detected by WES.

TABLE 6
Specific gene alterations associated with shorter PFS

| | | Cox model Wald | Log rank |
|---|---|---|---|
| Gene | HR (95% CI) | test P value | test P value |
| PIK3CA | 2.53 (1.2~5.34) | 0.0150 | 0.0121 |
| AKT3 | 3.83 (1.67~8.8) | 0.0015 | 0.0007 |
| DDR2 | 3.3 (1.41~7.72) | 0.0060 | 0.0035 |
| ESR1 | 3.78 (1.69~8.45) | 0.0012 | 0.0005 |
| ATM | 5.12 (2.2~11.94) | 0.0002 | 2.82E−05 |
| AURKA | 5.2 (2.06~13.13) | 0.0005 | 0.0001 |
| MUC16 | 3.07 (1.22~7.7) | 0.0170 | 0.0115 |
| BRCA2 | 4.25 (1.51~11.93) | 0.0061 | 0.0027 |
| CCND1 | 4.13 (1.63~10.45) | 0.0027 | 0.0012 |
| CCNE2 | 2.83 (1.06~7.58) | 0.0381 | 0.0311 |
| DSP | 5.39 (1.85~15.74) | 0.0021 | 0.0005 |
| MYC | 2.94 (1.01~8.59) | 0.0488 | 0.0404 |
| PLCG1 | 12.9 (4.05~41.04) | 0.0000 | 5.02E−08 |
| RB1 | 3.2 (1.04~9.85) | 0.0424 | 0.0307 |
| RUNX1T1 | 4.45 (1.6~12.35) | 0.0041 | 0.0018 |
| USH2A | 4.51 (1.61~12.65) | 0.0042 | 0.0018 |
| ZFHX3 | 4.27 (1.37~13.33) | 0.0125 | 0.0078 |

A comparison of the most frequently altered genes detected at progression versus baseline was made across all evaluable samples from patients who had progressed at the time of analysis (28/29) (FIG. 5A). The most frequently observed alterations across all 28 patients at baseline were PIK3CA (54%), TP53 (39%), AKT3 (32%), DDR2 (29%), ATM (29%), AURKA (25%) ESR1 (25%), BRCA2 (21%) and EGFR (21%), whereas at progression the most frequent alterations were TP53 (50%), PIK3CA (43%), RB1 (36%), AURKA (32%), CCND1 (32%), ESR1 (32%), BRCA2 (29%), ATM (25%), and MUC12 (25%). Alterations in RB1 were significantly enriched at progression (36% versus 14%) (P=0.04) (McNemar's Test) (FIG. 5B). Non-significant enrichment at progression versus baseline was observed for a number of additional alterations previously implicated in CDK4/6i and/or ET resistance including AR (18% versus 7%) AURKA (32% versus 25%), CCND1 (32% versus 18%), CDKN2A (21% versus 14%), ESR1 (32% versus 25%), FGFR1 (18% versus 14%), PTEN (21% versus 11%), MYC (18% versus 14%) and TP53 (50% versus 39%). Non-significant enrichment of additional alterations not commonly implicated in CDK4/6i and ET resistance was also observed at progression, including BRCA2 (29% versus 21%), CBL (18% versus 7%), CDH1 (18% versus 11%), KMT2D (18% versus 14%), MUC12 (25% versus 14%) and PREX2 (21% versus 14%) (FIG. 5B). Two of the enriched genes (MUC12 and PREX2) were not targeted by the PredicineATLAS™ panel, thereby representing novel alterations detected by WES. In contrast to increased levels of some individual variants, no significant difference was observed between the median bTMB or bCNB levels detected at baseline vs. progression (FIGS. 14A-14B). In summary, extension of boosted WES sequencing to blood samples collected at baseline and progression enabled the determination of additional candidate biomarkers of de novo and acquired resistance.

Further, we demonstrated that bCNB scores predict poor clinical outcomes and increase before radiographic detection of clinical progression. bCNB scores reflecting genomic-wide assessment of CNV were derived from LP-WGS data generated from all 51 baseline samples, 47 C1D15 samples, 51 C2D1 samples, 38 staging samples, and 29 progression samples (FIG. 1B). The median bCNB score at baseline was 9.36 [IQR 5.78-13.2). Baseline bCNB scores were significantly higher in patients who experienced progression within 6 months (without clinical benefit) (Wilcoxon Test, P=0.036) (FIG. 6A), and a high baseline bCNB score defined by a cutoff of 5.6 was significantly associated with shorter PFS (HR 3.14 [CI 1.34-7.74] (P=0.009) (FIG. 6B). Baseline bCNB scores were also significantly correlated with baseline bTMB scores (R=0.68, P=6.6×10⁻⁰⁸) (FIG. 15). Serial analysis of bCNB during treatment revealed decreases at C1D15 and/or C2D1 relative to baseline levels in 38/51 (74.5%) patients. Staging samples were collected concurrently with imaging studies performed every 3 months for assessment of tumor response. One to 3 timepoints (3 to 9 months) immediately prior to progression were available from 18 of the 29 patients who progressed. Analysis of these samples revealed increases (over previous nadir levels) that preceded imaging detection of progressive disease by at least 3 months in 12/18 (66.7%) of patients, as shown in FIG. 6C. An increase in bCNB preceded imaging detection of progressive disease by 6 months in 6 patients, and by 9 months in 1 patient. Comparison of dynamic bCNB patterns using the orthogonal measure of ctDNA fraction from PredicineATLAS+™ profiling in a subset of 4 patients revealed similar patterns with ctDNA fraction levels (FIG. 16). In summary, we showed that high baseline bCNB was significantly associated with poor clinical outcomes, and that increases in bCNB preceded radiographic detection of clinical progression in two thirds of the cases monitored over the course of treatment.

As described, we reported a comprehensive ctDNA NGS analysis, encompassing a plasma-based boosted WES assay, LP-WGS, and a bioinformatics pipeline for determining bTMB and bCNB, to enable a genome-wide evaluation of novel resistance mechanisms and clonal evolution in patients with HR+/HER2-MBC receiving ET in combination with CDK4/6i. Specifically, we identified a subset of patients, defined by hypermutation (high bTMB) and increased copy number variation (high bCNB), in association with poor outcomes who require novel therapeutic strategies. In addition, PredicineWES+™ enabled the expanded detection of genomic alterations associated with resistance at baseline and progression. We also demonstrated that dynamic changes in LP-WGS-derived bCNB scores over the course of treatment preceded radiographic response and clinical progression in a subset of patients, identifying potential utility for response monitoring. These studies using non-invasive blood-based sequencing represent a comprehensive evaluation of genome-wide ctDNA in this patient population, resulting in the generation of biological insights and a potential therapeutic hypothesis to improve clinical outcomes.

Importantly, bTMB and bCNB were determined using one 8-ml tube of whole blood in all patients with evaluable samples, indicating the feasibility from a clinical application standpoint. As expected, median bTMB was relatively low in this cohort (less than 2 MBp), a finding that may be consistent with evaluation of tTMB in breast cancer patients and particularly patients with HR+MBC [30]. Based on the observed association of high bTMB with lack of clinical benefit and shorter PFS, we demonstrated a stratification tool with treatment implications. (TMB may be predictive of response for patients treated with ICI monotherapy in other tumor types [20]. However, defining optimal cut points based on utilization of different sequencing platforms, bioinformatics techniques, and methods for determining tTMB may remain challenging. Therefore, it appears that optimal tTMB thresholds may vary across different tumor types [46]. Therefore we did not use an a priori bTMB threshold in the outcome analysis. Instead, multiple bTMB thresholds including the median (1.9 MBp), third quartile (3.8 MBp), and FDA-approved threshold of 10 MBp in tissue, were significantly associated with PFS. These findings reinforce the consistency of defining a hypermutated, resistant subset of patients with higher bTMB.

Interestingly, the high bTMB patients in our cohort were enriched for dominant APOBEC mutational signatures. The APOBEC family of DNA editing enzymes generate mutations during a variety of normal biologic processes including innate and adaptive immune responses [47]. However, upregulated “off target” activity of APOBEC enzymes may be a major source of somatic mutations in a number of cancers resulting in distinctive mutational signatures [22, 43-44]. APOBEC signatures may be observed in a variety of hypermutated malignancies and may be associated with response to ICI [30-31, 33, 48]. The observed enrichment of these signatures in high vs. low bTMB HR+/HER2-negative patients in this cohort further underscores the identification of a biomarker-defined subset of patients that may benefit from the incorporation of ICI therapy. Our study also identified several oncogenic pathways (e.g. Cell Cycle, DDR, NOTCH, PI3K, and RTK-RAS) associated with high bTMB as potential drug targets.

Our data also demonstrate an overlap between patients with high bTMB and endocrine resistance defined by ESMO criteria. While patients with clinically defined endocrine resistance had similar median bTMB compared to patients with de novo MBC or patients with endocrine sensitive disease, a majority of high bTMB cases were present in the endocrine resistant cohort at baseline. Importantly, bTMB scores stratified PFS in the subgroup of patients with clinically defined endocrine resistance. Moreover, patients with ESR1 mutations at baseline had higher bTMB scores compared to patients with wild-type ESR1. Clinically defined endocrine resistance, sites of metastatic disease on imaging, and other pathological variables did not stratify baseline patients with worse prognosis, further supporting the need for novel biomarkers for risk stratification. Collectively, these findings demonstrate the use of bTMB scores to define a subgroup of patients unlikely to respond to standard first-line therapy with CDK4/6i and ET, and these findings may be used to determine, and administer to patients, alternative combination treatment strategies including ICI.

Our findings indicate that novel treatment strategies are needed for high bTMB and high bCNB patients at baseline. The association of high tTMB with response to ICI based on the tissue agnostic approval of pembrolizumab for patients with high tTMB (defined at a threshold above 10 mutations/MBp) indicates a potential treatment approach [49]. In the TAPUR and NIMBUS studies, a subset of patients with MBC and high tTMB across subtypes were durable responders [32, 50]. However, in other non-biomarker selected populations, there has been no improvement in outcomes when adding ICI to chemotherapy [51]. For this reason, evaluating the potential of incorporating ICI for HR+/HER2-negative patients with high bTMB, either as monotherapy or in combination, is needed. Preclinical data indicate that CDK4/6i enhances T-cell activation, increases tumor infiltration, and may have a synergistic effect with ICI therapy [52]. While chemotherapy for patients with HR+HER2 negative MBC may be reserved for impending organ failure or endocrine refractory disease, the optimal use of chemotherapy in this biologically defined cohort may be explored, and these patients may benefit from earlier incorporation of cytotoxic therapy. In addition, because of the potential for bCNB to precede clinical detection of disease recurrence, interventional studies may be performed to determine whether early switching of therapy based on molecular progression of disease, as opposed to imaging progression, may improve clinical outcomes.

Using a genome-wide approach also identified many individual resistance alterations, validating previously implicated mechanisms and yielding discovery of novel candidate genes. Baseline alterations in RB1 and other genes associated with de novo resistance to ET+CDK4/6i therapy, were determined to be associated with shorter PFS, as were novel baseline alterations in DSP, MUC16, PLCGI, USH2A and ZFHX3. Although median levels of bTMB and bCNB were not significantly increased at the time of clinical progression, we observed enrichment of individual alterations previously implicated in endocrine and/or CDK4/6i treatment resistance including AR, AURKA, CCND1, CDKN2A, ESR1, FGFR1, MYC and RB1 [53]. We also observed enrichment of alterations in genes less commonly associated with ET and CDK4/6i treatment resistance, which encode a variety of oncogenic proteins, including CBL, a member of the RING finger ubiquitin ligase family that regulates receptor tyrosine kinase signaling [54-56]; KMT2D, a methyltransferase involved in estrogen receptor recruitment and activation [57]; MUC12, a glycosylated transmembrane protein in the mucin family implicated in the regulation of proliferation, invasion and metastatic potential [58-60]; and PREX2, a guanine nucleotide exchange factor that regulates cancer cell motility and invasion [61-62]. Many of these novel alterations are not covered by targeted sequencing panels, underscoring the value of the extended WES to identify diverse mechanisms of resistance.

Performing WES extended the gold standard of TMB measurement to blood samples and enabled the discovery of novel candidate resistance mechanisms, which may further enable clinical applications such as administering appropriate therapeutics. However, we observed high correlation between bTMB measurements obtained by WES and shorter targeted sequencing panels, demonstrating the utility of measuring bTMB in the clinic using cost-effective tests. Further, we demonstrated that bCNB, derived from more cost-effective PredicineCNB™, illustrated a high degree of concordance with bTMB at baseline and was also associated with poor patient outcomes. We also demonstrated the utility of serial bCNB evaluation for monitoring dynamic changes in ctDNA during treatment. bCNB declined as early as two weeks after treatment initiation, thereby providing an early signal of molecular response to therapy (e.g., earlier than detectable by imaging). In addition, when comparing concurrent imaging and bCNB assessment, an increase in bCNB preceded clinical progression of disease in two-thirds of patients. Therefore, serial blood-based molecular assessments may serve as a surrogate for PFS, which may be clinically assessed via imaging.

Because some patients on study had not progressed, the landscape of alterations at progression may be explored to evaluate the extent to which it is reflective of patients with long-term response to therapy. Also, an alternative dosing regimen of Palbociclib was used, which may affect the development of resistance alterations. Further, concurrent blood and tissue biopsies may be performed for TMB testing.

In summary, our study demonstrates the potential utility of blood based bCNB and bTMB assessment in treatment decision making for patients with HR+/HER2-negative MBC. Furthermore, this study demonstrates the utility of performing whole-genome ctDNA analysis to comprehensively define the molecular mechanisms of baseline and serial resistance to CDK4/6i combined with ET for patients with HR+HER2 negative MBC. The results identified a subset of patients at baseline with poor outcome to standard-of-care first-line therapy and also demonstrates a non-invasive approach for detecting early blood-based progression. Using bTMB and bCNB results, optimal treatments may be selected for these hypermutated, genomically complex patients, such as the incorporation of early ICI and combination therapy.

### REFERENCES

1. Sledge G W, Jr., Toi M, Neven P et al. The Effect of Abemaciclib Plus Fulvestrant on Overall Survival in Hormone Receptor-Positive, ERBB2-Negative Breast Cancer That Progressed on Endocrine Therapy-MONARCH 2: A Randomized Clinical Trial. JAMA Oncol 2020; 6:116-124 is incorporated by reference in its entirety.
2. Giuliano M, Schettini F, Rognoni C et al. Endocrine treatment versus chemotherapy in postmenopausal women with hormone receptor-positive, HER2-negative, metastatic breast cancer: a systematic review and network meta-analysis. Lancet Oncol 2019; 20:1360-1369 is incorporated by reference in its entirety.
3. Turner N C, Slamon D J, Ro J et al. Overall Survival with Palbociclib and Fulvestrant in Advanced Breast Cancer. N Engl J Med 2018; 379:1926-1936 is incorporated by reference in its entirety.
4. Im S A, Lu Y S, Bardia A et al. Overall Survival with Ribociclib plus Endocrine Therapy in Breast Cancer. N Engl J Med 2019; 381:307-316 is incorporated by reference in its entirety.
5. Slamon D J, Neven P, Chia S et al. Overall Survival with Ribociclib plus Fulvestrant in Advanced Breast Cancer. N Engl J Med 2020; 382:514-524 is incorporated by reference in its entirety.
6. Turner N C, Kingston B, Kilburn L S et al. Circulating tumour DNA analysis to direct therapy in advanced breast cancer (plasmaMATCH): a multicentre, multicohort, phase 2a, platform trial. Lancet Oncol 2020; 21:1296-1308 is incorporated by reference in its entirety.
7. Alix-Panabieres C, Pantel K. Clinical Applications of Circulating Tumor Cells and Circulating Tumor DNA as Liquid Biopsy. Cancer Discov 2016; 6:479-491 is incorporated by reference in its entirety.
8. Davis A A, Jacob S, Gerratana L et al. Landscape of circulating tumour DNA in metastatic breast cancer. EBioMedicine 2020; 58:102914 is incorporated by reference in its entirety.
9. Wan J C M, Massie C, Garcia-Corbacho J et al. Liquid biopsies come of age: towards implementation of circulating tumour DNA. Nat Rev Cancer 2017; 17:223-238 is incorporated by reference in its entirety.
10. O'Leary B, Cutts R J, Liu Y et al. The Genetic Landscape and Clonal Evolution of Breast Cancer Resistance to Palbociclib plus Fulvestrant in the PALOMA-3 Trial. Cancer Discov 2018; 8:1390-1403 is incorporated by reference in its entirety.
11. Andre F, Ciruelos E, Rubovszky G et al. Alpelisib for PIK3CA-Mutated, Hormone Receptor-Positive Advanced Breast Cancer. N Engl J Med 2019; 380:1929-1940 is incorporated by reference in its entirety.
12. FDA approves alpelisib for metastatic breast cancer. May 24, 2019. https://www.fda.gov/drugs/resources-information-approved-drugs/fda-approves-alpelisib-metastatic-breast-cancer is incorporated by reference in its entirety.
13. Turner N C, Liu Y, Zhu Z et al. Cyclin El Expression and Palbociclib Efficacy in Previously Treated Hormone Receptor-Positive Metastatic Breast Cancer. J Clin Oncol 2019; 37:1169-1178 is incorporated by reference in its entirety.
14. Li Z, Razavi P, Li Q et al. Loss of the FAT1 Tumor Suppressor Promotes Resistance to CDK4/6 Inhibitors via the Hippo Pathway. Cancer Cell 2018; 34:893-905 e898 is incorporated by reference in its entirety.
15. Formisano L, Lu Y, Servetto A et al. Aberrant FGFR signaling mediates resistance to CDK4/6 inhibitors in ER+ breast cancer. Nat Commun 2019; 10:1373 is incorporated by reference in its entirety.
16. Turner N, Pearson A, Sharpe R et al. FGFR1 amplification drives endocrine therapy resistance and is a therapeutic target in breast cancer. Cancer Res 2010; 70:2085-2094 is incorporated by reference in its entirety.
17. Costa C, Wang Y, Ly A et al. PTEN Loss Mediates Clinical Cross-Resistance to CDK4/6 and PI3Kalpha Inhibitors in Breast Cancer. Cancer Discov 2020; 10:72-85 is incorporated by reference in its entirety.
18. Condorelli R, Spring L, O'Shaughnessy J et al. Polyclonal RB1 mutations and acquired resistance to CDK 4/6 inhibitors in patients with metastatic breast cancer. Ann Oncol 2018; 29:640-645 is incorporated by reference in its entirety.
19. Fancello L, Gandini S, Pelicci P G, Mazzarella L. Tumor mutational burden quantification from targeted gene panels: major advancements and challenges. J Immunother Cancer 2019; 7:183 is incorporated by reference in its entirety.
20. Rizvi N A, Hellmann M D, Snyder A et al. Cancer immunology. Mutational landscape determines sensitivity to PD-1 blockade in non-small cell lung cancer. Science 2015; 348:124-128 is incorporated by reference in its entirety.
21. Snyder A, Makarov V, Merghoub T et al. Genetic basis for clinical response to CTLA-4 blockade in melanoma. N Engl J Med 2014; 371:2189-2199 is incorporated by reference in its entirety.
22. Alexandrov L B, Nik-Zainal S, Wedge D C et al. Signatures of mutational processes in human cancer. Nature 2013; 500:415-421 is incorporated by reference in its entirety.
23. Chalmers Z R, Connelly C F, Fabrizio D et al. Analysis of 100,000 human cancer genomes reveals the landscape of tumor mutational burden. Genome Med 2017; 9:34 is incorporated by reference in its entirety.
24. Yarchoan M, Albacker L A, Hopkins A C et al. PD-L1 expression and tumor mutational burden are independent biomarkers in most cancers. JCI Insight 2019; 4 is incorporated by reference in its entirety.
25. Gandara D R, Paul S M, Kowanetz M et al. Blood-based tumor mutational burden as a predictor of clinical benefit in non-small-cell lung cancer patients treated with atezolizumab. Nat Med 2018; 24:1441-1448 is incorporated by reference in its entirety.
26. Wang Z, Duan J, Cai S et al. Assessment of Blood Tumor Mutational Burden as a Potential Biomarker for Immunotherapy in Patients With Non-Small Cell Lung Cancer With Use of a Next-Generation Sequencing Cancer Gene Panel. JAMA Oncol 2019; 5:696-702 is incorporated by reference in its entirety.
27. Kim E S, Velcheti V, Mekhail T et al. Blood-based tumor mutational burden as a biomarker for atezolizumab in non-small cell lung cancer: the phase 2 B-FIRST trial. Nat Med 2022 is incorporated by reference in its entirety.
28. Bos M K, Angus L, Nasserinejad K et al. Whole exome sequencing of cell-free DNA-A systematic review and Bayesian individual patient data meta-analysis. Cancer Treat Rev 2020; 83:101951 is incorporated by reference in its entirety.
29. Koeppel F, Blanchard S, Jovelet C et al. Whole exome sequencing for determination of tumor mutation load in liquid biopsy from advanced cancer patients. PLOS One 2017; 12: e0188174 is incorporated by reference in its entirety.
30. Barroso-Sousa R, Jain E, Cohen O et al. Prevalence and mutational determinants of high tumor mutation burden in breast cancer. Ann Oncol 2020; 31:387-394 is incorporated by reference in its entirety.
31. Wang R, Yang Y, Ye W W et al. Case Report: Significant Response to Immune Checkpoint Inhibitor Camrelizumab in a Heavily Pretreated Advanced ER+/HER2-Breast Cancer Patient With High Tumor Mutational Burden. Front Oncol 2020; 10:588080 is incorporated by reference in its entirety.
32. Barroso-Sousa R, Li T, Reddy S et al. Abstract GS2-10: Nimbus: A phase 2 trial of nivolumab plus ipilimumab for patients with hypermutated her2-negative metastatic breast cancer (MBC). Cancer Research. February 2022 is incorporated by reference in its entirety
33. Wang S, Jia M, He Z, Liu X S. APOBEC3B and APOBEC mutational signature as potential predictive markers for immunotherapy response in non-small cell lung cancer. Oncogene 2018; 37:3924-3936 is incorporated by reference in its entirety.
34. Dietlein F, Weghorn D, Taylor-Weiner A et al. Identification of cancer driver genes based on nucleotide context. Nat Genet 2020; 52:208-218 is incorporated by reference in its entirety.
35. Martinez-Jimenez F, Muinos F, Sentis I et al. A compendium of mutational cancer driver genes. Nat Rev Cancer 2020; 20:555-572 is incorporated by reference in its entirety.
36. Dawson S J, Tsui D W, Murtaza M et al. Analysis of circulating tumor DNA to monitor metastatic breast cancer. N Engl J Med 2013; 368:1199-1209 is incorporated by reference in its entirety.
37. O'Leary B, Cutts R J, Huang X et al. Circulating Tumor DNA Markers for Early Progression on Fulvestrant With or Without Palbociclib in ER+ Advanced Breast Cancer. J Natl Cancer Inst 2021; 113:309-317 is incorporated by reference in its entirety.
38. Jacob S, Davis A A, Gerratana L et al. The use of serial circulating tumor DNA (ctDNA) to detect resistance alterations in progressive metastatic breast cancer. Clin Cancer Res 2020 is incorporated by reference in its entirety.
39. Davis A A, Iams W T, Chan D et al. Early Assessment of Molecular Progression and Response by Whole-genome Circulating Tumor DNA in Advanced Solid Tumors. Mol Cancer Ther 2020; 19:1486-1496 is incorporated by reference in its entirety.
40. Jongbloed E M, Deger T, Sleijfer S et al. A Systematic Review of the Use of Circulating Cell-Free DNA Dynamics to Monitor Response to Treatment in Metastatic Breast Cancer Patients. Cancers (Basel) 2021; 13 is incorporated by reference in its entirety.
41. Krishnamurthy J, Luo J, Suresh R et al. A phase II trial of an alternative schedule of palbociclib and embedded serum TKI analysis. NPJ Breast Cancer 2022; 8:35 is incorporated by reference in its entirety.
42. Cardoso F, Paluch-Shimon S, Senkus E et al. 5th ESO-ESMO international consensus guidelines for advanced breast cancer (ABC 5). Ann Oncol 2020; 31:1623-1649 is incorporated by reference in its entirety.
43. Alexandrov L B, Kim J, Haradhvala N J et al. The repertoire of mutational signatures in human cancer. Nature 2020; 578:94-101 is incorporated by reference in its entirety.
44. Granadillo Rodriguez M, Flath B, Chelico L. The interesting relationship between APOBEC3 deoxycytidine deaminases and cancer: a long road ahead. Open Biol 2020; 10:200188 is incorporated by reference in its entirety.
45. Sanchez-Vega F, Mina M, Armenia J et al. Oncogenic Signaling Pathways in The Cancer Genome Atlas. Cell 2018; 173:321-337 e310 is incorporated by reference in its entirety.
46. Samstein R M, Lee C H, Shoushtari A N et al. Tumor mutational load predicts survival after immunotherapy across multiple cancer types. Nat Genet 2019; 51:202-206 is incorporated by reference in its entirety.
47. Knisbacher B A, Gerber D, Levanon E Y. DNA Editing by APOBECs: A Genomic Preserver and Transformer. Trends Genet 2016; 32:16-28 is incorporated by reference in its entirety.
48. Miao D, Margolis C A, Vokes N I et al. Genomic correlates of response to immune checkpoint blockade in microsatellite-stable solid tumors. Nat Genet 2018; 50:1271-1281 is incorporated by reference in its entirety.
49. Marabelle A, Le D T, Ascierto P A et al. Efficacy of Pembrolizumab in Patients With Noncolorectal High Microsatellite Instability/Mismatch Repair-Deficient Cancer: Results From the Phase II KEYNOTE-158 Study. J Clin Oncol 2020; 38:1-10 is incorporated by reference in its entirety.
50. Alva A S, Mangat P K, Garrett-Mayer E et al. Pembrolizumab in Patients With Metastatic Breast Cancer With High Tumor Mutational Burden: Results From the Targeted Agent and Profiling Utilization Registry (TAPUR) Study. J Clin Oncol 2021; 39:2443-2451 is incorporated by reference in its entirety.
51. Keenan T E, Guerriero J L, Barroso-Sousa R et al. Molecular correlates of response to eribulin and pembrolizumab in hormone receptor-positive metastatic breast cancer. Nat Commun 2021; 12:5563 is incorporated by reference in its entirety.
52. Deng J, Wang E S, Jenkins R W et al. CDK4/6 Inhibition Augments Antitumor Immunity by Enhancing T-cell Activation. Cancer Discov 2018; 8:216-233 is incorporated by reference in its entirety.
53. Asghar U S, Kanani R, Roylance R, Mittnacht S. Systematic Review of Molecular Biomarkers Predictive of Resistance to CDK4/6 Inhibition in Metastatic Breast Cancer. JCO Precis Oncol 2022; 6: e2100002 is incorporated by reference in its entirety.
54. Daniels S R, Liyasova M, Kales S C et al. Loss of function Cbl-c mutations in solid tumors. PLOS One 2019; 14: e0219143 is incorporated by reference in its entirety.
55. Wang Y, Dai J, Zeng Y et al. E3 Ubiquitin Ligases in Breast Cancer Metastasis: A Systematic Review of Pathogenic Functions and Clinical Implications. Front Oncol 2021; 11:752604 is incorporated by reference in its entirety.
56. Xu L, Zhang Y, Qu X et al. E3 Ubiquitin Ligase Cbl-b Prevents Tumor Metastasis by Maintaining the Epithelial Phenotype in Multiple Drug-Resistant Gastric and Breast Cancer Cells. Neoplasia 2017; 19:374-382 is incorporated by reference in its entirety.
57. Toska E, Osmanbeyoglu H U, Castel P et al. PI3K pathway regulates ER-dependent transcription in breast cancer through the epigenetic regulator KMT2D. Science 2017; 355:1324-1330 is incorporated by reference in its entirety.
58. Gao S L, Yin R, Zhang L F et al. The oncogenic role of MUC12 in RCC progression depends on c-Jun/TGF-beta signalling. J Cell Mol Med 2020; 24:8789-8802 is incorporated by reference in its entirety.
59. Mukhopadhyay P, Chakraborty S, Ponnusamy M P et al. Mucins in the pathogenesis of breast cancer: implications in diagnosis, prognosis and therapy. Biochim Biophys Acta 2011; 1815:224-240 is incorporated by reference in its entirety.
60. van Putten J P M, Strijbis K. Transmembrane Mucins: Signaling Receptors at the Intersection of Inflammation and Cancer. J Innate Immun 2017; 9:281-299 is incorporated by reference in its entirety.
61. Mense S M, Barrows D, Hodakoski C et al. PTEN inhibits PREX2-catalyzed activation of RACI to restrain tumor cell invasion. Sci Signal 2015; 8: ra32 is incorporated by reference in its entirety.
62. Pandiella A, Montero J C. Molecular pathways: P-Rex in cancer. Clin Cancer Res 2013; 19:4564-4569 is incorporated by reference in its entirety.

### Supplementary Methods

Blood collection and cfDNA/gDNA extraction were performed as follows. Each single draw of 8 mL of whole blood was collected into a Streck tube, and then two-step centrifugation was performed to separate plasma and buffy coat compartments. Aliquoted samples were stored at −80° C. for batch processing. Circulating cell-free DNA (cfDNA) was extracted from plasma samples using the QIAamp circulating nucleic acid kit (Qiagen, Hilden, Germany). Quantity and quality of the purified cfDNA were checked using a Qubit fluorimeter (ThermoFisher Scientific, Waltham, Massachusetts, USA) and Bioanalyzer 2100 (Agilent Technologies, California, USA. For cfDNA samples with severe genomic contamination from peripheral blood cells, a bead-based size selection was performed to remove large genomic fragments (AMPure XP beads, Beckman Coulter, California, USA). Genomic DNA (gDNA) was extracted from matched peripheral blood mononuclear cells (PBMCs) using the QIAamp DNA Blood Mini Kit (Qiagen), then enzymatically fragmented and purified.

Library preparation, hybrid capture and sequencing were performed as follows. Five to 30 ng of extracted cfDNA or 30-50 ng of fragmented PBMC gDNA were processed for library construction including end-repair dA-tailing and adapter ligation. Ligated library fragments with appropriate adapters were amplified via PCR. The amplified DNA libraries were then further checked using a Bioanalyzer 2100 and samples with sufficient yield were advanced to hybrid capture.

Hybrid capture was conducted using Biotin labelled DNA probes. In brief, each library was hybridized overnight with a Predicine NGS panel and paramagnetic beads. The unbound fragments were washed away, and the enriched fragments were amplified via PCR amplification. The purified product was checked on a Bioanalyzer 2100 and then loaded into an Illumina NovaSeq 6000 (San Diego, CA, USA) for NGS sequencing with paired-end 2×150 bp sequencing kits.

Analyses of NGS data from cfDNA were performed as follows. NGS data from cfDNA were analyzed using the Predicine DeepSea NGS analysis pipeline, which starts from the raw sequencing data (BCL files) and outputs the final mutation calls. Briefly, the pipeline first performed adapter trimming, barcode checking, and correction. Cleaned paired FASTQ files were aligned to human reference genome build hg19 using the BWA alignment tool. Consensus bam files were then derived by merging paired-end reads originated from the same molecules (based on mapping location and unique molecular identifiers) as single strand fragments. Single strand fragments from the same double strand DNA molecules were further merged as double stranded. By performing error suppression (e.g., as described by [Newman, 2016]), both sequencing and PCR errors were mostly corrected during this process.

Somatic mutation identification was performed as follows. Candidate variants were called by comparing with local variant background (defined based on plasma samples from healthy donors and historical data). Variants were further filtered by log-odds (LOD) threshold [Cibulskis 2013], base and mapping quality thresholds, repeat regions and other quality metrics. In general, a variant identified in cfDNA was considered a candidate somatic mutation (i) only when at least three distinct fragments (one of them should be double-stranded) contained the mutation, (ii) the mutation allele frequency was higher than 0.25%, or 0.1% for hotspot mutations, and (iii) the ctDNA variant fragments were significantly over-represented in comparison with the matched PBMC sample using Fisher's Exact test.

Candidate somatic mutations were further filtered on the basis of gene annotation to identify those occurring in protein-coding regions. Intronic and silent changes were excluded, while mutations resulting in missense mutations, nonsense mutations, frameshifts, or splice site alterations were retained. Mutations annotated as benign or likely benign were also filtered out based on the ClinVar database, or as common germline variants in databases including 1000 genomes, ExAC, gnomAD and KAVIAR with population allele frequency >0.5%. Finally, hematopoietic expansion-related variants that have been previously described, including those in DNMT3A, ASXL1, TET2, and specific alterations within ATM (residue 3008), GNAS (residue 201, 202), or JAK2 (residue 617) were marked as CHIP-related mutations.

Germline DNA analysis was performed as follows. Germline variants were determined by concurrent sequencing of buffy coat PBMCs. Candidate variants with low base quality, mapping scores, and other poor quality metrics were filtered. Candidate variants with an allelic frequency <5% or with less than 8 distinct reads containing the mutation were excluded. Unknown variants in repeat regions were also excluded. Details of the analytical workflow are provided above in “Analyses of NGS data generated from cfDNA”.

Copy number analysis by targeted panel was performed as follows. Copy number variations were estimated at the gene level. The pipeline calculates the on-target unique fragment coverage based on consensus bam files, which is first corrected for GC bias, and is then adjusted for the probe level bias (estimated from a pooled reference). Each adjusted coverage profile is self-normalized (assuming a diploid status of each sample) first, and then compared against correspondingly adjusted coverages from a group of normal reference samples to estimate the significance of the copy number variant. To call an amplification or deletion of a gene requires the absolute z-score and copy number change pass minimum thresholds.

Analysis of DNA re-arrangements was performed as follows. DNA re-arrangement was detected by identifying the alignment breaking points based on the bam files before the consensus step. Suspicious alignments were filtered based on repeat regions, local entropy calculation and similarity between reference and alternative alignments. Larger than 3 unique alignments (at least one of them should be double stranded) are required to report a DNA fusion.

Analysis of ctDNA fraction was performed as follows. ctDNA fractions were estimated based on the allele fractions of autosomal somatic mutations (e.g., as described by [Vandekerkhove, 2017]). Briefly, the mutant allele fraction (MAF) and ctDNA fraction are related as MAF=(ctDNA*1)/[(1−ctDNA)*2+ctDNA*1], and thus ctDNA=2/((1/MAF)+1). Somatic mutations in genes with a detectable copy number change are omitted from ctDNA fraction estimation.

bTMB score estimation was performed as follows. Blood-based tumor mutational burden (bTMB) was defined as the number of somatic coding SNVs including synonymous and nonsynonymous variants within panel target regions. The bTMB score was then normalized by the total effective targeted panel size within the coding region [Gandara, 2018]. Because TMB estimation considers all variants (including synonymous and non-whitelist variants), higher variant call specificity is required for TMB estimation. As a result, more stringent cut-offs were used for variants calls, and only variants with allele frequency >0.35% were used in TMB estimation. Samples with the maximum somatic allelic frequency (MSAF)<0.7% were excluded for bTMB estimation. Variants in common CHIP genes (DNMT3A, TET2, ASXL1 and JAK2) were excluded in TMB estimation.

Copy number burden analysis using low-pass whole genome sequencing was performed as follows. Low-pass whole genome sequencing (LP-WGS) with an overall average coverage of 5× was performed on patient samples. The ichorCNA algorithm [Adalsteinsson, 2017] was applied to GC and mappability-normalized reads to estimate plasma copy number variations using the hidden Markov model (HMM). First, we measured segment level (1 Mb genomic regions) copy number deviation as log 2 ratio of the normalized reads between a sample and the average of a group of normal plasma samples as background. Then we quantified arm-level CNV deviation as the average of segment CNVs across each chromosome arm. Finally, we calculated sample level copy number burden (CNB score) as the sum of absolute z-score of arm-level CNV deviations, where a higher/lower CNB score indicates higher/lower CNV abnormality compared with normal background. The CNB score cutoff of 5.6 was defined as three standard deviations away from the population mean of normal plasma CNB scores.

Mutational signature analysis was performed as follows. Mutational signature analysis was performed to compare patterns of single base substitutions (SBS) with previously reported SBS signatures [Alexandrov, 2013, 2020] now available in the COSMIC data base using the maftools package (version 2.4.15) in R (version 3.6.3). Briefly, each of the 96 possible mutation substitution types is defined by one of six substitution types (T>A, T>C, T>G, C>A, C>G, C>T) and the bases immediately 5′ and 3′ to the mutated base. For each sample, the number of mutation substitutions was counted. Non-negative matrix factorization was used to decompose the count matrix into n signatures. The number of signatures (n) best fitting the data was estimated using Cophenetic correlation. Signatures were compared to the 78 available SBS COSMIC signatures (v3.2-March 2021, cancer.sanger.ac.uk/signatures/sbs/) using cosine similarity. A dominant signature was defined as the signature with the maximum signature score in each sample.

Oncogenic signaling pathway analysis was performed as follows. To compare the relative proportion of mutations within key oncogenic signaling pathways in high vs. low bTMB patients, we filtered a list of genes describing oncogenic signaling pathways [Sanchez-Vega, 2018] to include only those identified as breast cancer driver genes [Dietlein, 2020; Martinez-Jiminez, 2020]. The list of resulting genes is shown in the Table 7. The frequency of SNVs across these genes was compared between high vs. low bTMB patients and statistical significance was evaluated using the Fisher's Exact Test.

TABLE 7
Genes describing oncogenic signaling pathways
that are breast cancer driver genes

| Pathway | Gene List |
|---|---|
| HRD | BRCA1, BRCA2 |
| DDR | BRCA1, TP53, BAP1, BRCA2, PTEN, ATM, ARID1A, |
| | POLD1 |
| PI3K | PIK3CA, AKT1, PTEN, PIK3R1, MTOR |
| Cell_Cycle | RB1, CDKN2A, CDKN1B |
| TP53 | TP53, ATM |
| RTK-RAS | EGFR, FGFR2, BRAF, NF1, KRAS, ERBB3, ERBB4, |
| | JAK2, ALK, PDGFRB, HRAS, ERBB2 |
| Hippo | FAT1, FAT4, FAT3 |
| NOTCH | NOTCH2, CREBBP, NCOR2, FBXW7, SPEN |
| WNT | |
| MYC | |
| NRF2 | |
| TGF-Beta | SMAD2, SMAD4 |

### REFERENCES

1. Patel, P. G., et al., Preparation of Formalin-fixed Paraffin-embedded Tissue Cores for both RNA and DNA Extraction. J Vis Exp, 2016 (114) is incorporated by reference in its entirety.
2. Newman, A. M., et al., Integrated digital error suppression for improved detection of circulating tumor DNA. Nat Biotechnol, 2016. 34 (5): p. 547-555 is incorporated by reference in its entirety.
3. Cibulskis, K., et al., Sensitive detection of somatic point mutations in impure and heterogeneous cancer samples. Nat Biotechnol, 2013. 31 (3): p. 213-9 is incorporated by reference in its entirety.
4. Vandekerkhove, G., et al., Circulating Tumor DNA Reveals Clinically Actionable Somatic Genome of Metastatic Bladder Cancer. Clin Cancer Res, 2017. 23 (21): p. 6487-6497 is incorporated by reference in its entirety.
5. Gandara, D. R., et al., Blood-based tumor mutational burden as a predictor of clinical benefit in non-small-cell lung cancer patients treated with atezolizumab. Nat Med, 2018. 24 (9): p. 1441-1448 is incorporated by reference in its entirety.
6. Adalsteinsson, V. A., et al., Scalable whole-exome sequencing of cell-free DNA reveals high concordance with metastatic tumors. Nat Commun, 2017. 8 (1): p. 1324 is incorporated by reference in its entirety.
7. Alexandrov, L. B., et al., The repertoire of mutational signatures in human cancer. Nature, 2020. 578 (7793): p. 94-101 is incorporated by reference in its entirety.
8 Alexandrov, L. B., et al., Signatures of mutational processes in human cancer. Nature, 2013. 500 (7463): p. 415-21 is incorporated by reference in its entirety.
9 Sanchez-Vega, F., et al., Oncogenic Signaling Pathways in The Cancer Genome Atlas. Cell, 2018. 173 (2): p. 321-337 e10 is incorporated by reference in its entirety.
10. Dietlein, F., et al., Identification of cancer driver genes based on nucleotide context. Nat Genet, 2020. 52 (2): p. 208-218 is incorporated by reference in its entirety.
11. Martinez-Jimenez, F., et al., A compendium of mutational cancer driver genes. Nat Rev Cancer, 2020. 20 (10): p. 555-572 is incorporated by reference in its entirety.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-84. (canceled)

85. A method comprising:
(a) obtaining or deriving a biological sample from a subject, wherein said subject has cancer, has previously had cancer, or is suspected of having cancer;
(b) assaying cell-free deoxyribonucleic acid (cfDNA) molecules obtained or derived from said biological sample, wherein said assaying comprises sequencing at least a portion of said cfDNA molecules or derivatives thereof to produce a set of sequencing reads, wherein said sequencing comprises at least one of whole-exome sequencing (WES) and whole-genome sequencing (WGS); and
(c) determining at least one of a tumor mutational burden and a copy number burden of said subject, based at least in part on processing said set of sequencing reads.

86. The method of claim 85, wherein said biological sample is selected from the group consisting of: a plasma sample, a serum sample, a buffy coat sample, a urine sample, a saliva sample, a tissue biopsy sample, a pleural fluid sample, a peritoneal fluid sample, an amniotic fluid sample, a cerebrospinal fluid sample, a lymphatic fluid sample, a sweat sample, a tears sample, a semen sample, a derivative thereof, and a combination thereof.

87. The method of claim 85, wherein said biological sample is obtained or derived from said subject using an ethylenediaminetetraacetic acid (EDTA) collection tube, a cell-free deoxyribonucleic acid (DNA) collection tube, another blood collection tube, or a circulating tumor cell (CTC) collection tube.

88. The method of claim 85, further comprising subjecting said biological sample to conditions that are sufficient to isolate, enrich, or extract said cfDNA molecules.

89. The method of claim 85, further comprising fractionating a whole blood sample of said subject to obtain said cfDNA molecules.

90. The method of claim 85, wherein said WGS further comprises low-pass WGS.

91. The method of claim 85, wherein said sequencing in (b) further comprises next-generation sequencing, low-pass sequencing, targeted sequencing, methylation-aware sequencing, bisulfite sequencing, or a combination thereof.

92. The method of claim 85, wherein (b) further comprises amplifying at least a portion of said cfDNA molecules, genomic DNA (gDNA) molecules, or derivatives thereof.

93. The method of claim 92, wherein said amplifying further comprises polymerase chain reaction (PCR) or isothermal amplification.

94. The method of claim 85, wherein (b) further comprises using a microarray.

95. The method of claim 85, wherein said cancer is selected from the group consisting of: breast cancer, lung cancer, prostate cancer, colorectal cancer, melanoma, bladder cancer, non-Hodgkin lymphoma, kidney cancer, endometrial cancer, leukemia, pancreatic cancer, thyroid cancer, liver cancer, and a combination thereof.

96. The method of claim 95, wherein said cancer comprises said breast cancer.

97. The method of claim 96, wherein said breast cancer is metastatic breast cancer, hormone receptor-positive (HR+) breast cancer, HER2-negative (HER2−) breast cancer, or a combination thereof.

98. The method of claim 85, wherein said processing in (c) further comprises using a trained machine learning algorithm.

99. The method of claim 98, wherein said trained machine learning algorithm further comprises a deep learning algorithm, a support vector machine (SVM), a neural network, or a Random Forest.

100. The method of claim 85, further comprising, based at least in part on said at least one of said tumor mutational burden and said copy number burden of said subject, determining a relapse or recurrence of said cancer of said subject, or a resistance of said cancer to a drug treatment, a prognosis of said cancer of said subject.

101. The method of claim 100, wherein said prognosis comprises a likelihood of progression-free survival, a length of time for progression-free survival, a likelihood of overall survival, a length of time for overall survival, or a combination thereof.

102. The method of claim 85, wherein (a) further comprises obtaining or deriving said biological sample from said subject, (i) prior to said subject receiving a clinical intervention for said cancer, (ii) while the subject is receiving a clinical intervention for said cancer, (iii) subsequent to said subject receiving a clinical intervention for said cancer, or a combination thereof.

103. The method of claim 102, wherein said clinical intervention is selected from the group consisting of: surgical resection, chemotherapy, radiotherapy, immunotherapy, cell therapy, adjuvant therapy, neoadjuvant therapy, androgen deprivation therapy, and a combination thereof.

104. The method of claim 85, further comprising administering a clinical intervention to said subject, based at least in part on said at least one of said tumor mutational burden and said copy number burden of said subject.

105. The method of claim 104, wherein said clinical intervention is selected from the group consisting of: surgical resection, chemotherapy, radiotherapy, immunotherapy, endocrine therapy, adjuvant therapy, neoadjuvant therapy, androgen deprivation therapy, and a combination thereof.

106. The method of claim 85, wherein said set of sequencing reads comprises quantitative measures of a set of cancer-associated genomic loci.

107. The method of claim 106, wherein said set of cancer-associated genomic loci comprises one or more members selected from the group consisting of genes listed in Table 3, genes listed in Table 4, genes listed in Table 6, and genes listed in Table 7.

108. The method of claim 85, wherein (b) further comprises using nucleic acid primers or probes configured to selectively enrich said biological sample for DNA molecules corresponding to a set of genomic loci.

109. The method of claim 85, further comprising monitoring at least one of said tumor mutational burden and said copy number burden of said subject, wherein said monitoring comprises assessing, at each of a plurality of time points, said at least one of said tumor mutational burden and said copy number burden of said subject.

110. The method of claim 85, wherein said processing in (c) further comprises detecting tumor-associated alterations selected from the group consisting of: copy number alterations (CNAs), copy number losses (CNLs), single nucleotide variants (SNVs), insertions or deletions (indels), and rearrangements.

111. The method of claim 85, further comprising:
filtering at least a subset of said set of sequencing reads based on a quality score;
performing error correction on said set of sequencing reads using sample barcodes or molecular barcodes attached to at least one of said cfDNA molecules; or
performing at least one of single-stranded consensus calling and double-stranded consensus calling on said set of sequencing reads, thereby suppressing sequencing and PCR errors in said set of sequencing reads.

112. The method of claim 85, further comprising determining a mutant allele frequency of a set of somatic mutations.

113. A system comprising one or more computer processors and computer memory coupled thereto, the computer memory comprising machine executable code that, upon execution by the one or more computer processors, implements a method comprising:
(a) obtaining or deriving a biological sample from a subject, wherein said subject has cancer, has previously had cancer, or is suspected of having cancer;
(b) assaying cell-free deoxyribonucleic acid (cfDNA) molecules obtained or derived from said biological sample, wherein said assaying comprises sequencing at least a portion of said cfDNA molecules or derivatives thereof to produce a set of sequencing reads, wherein said sequencing comprises at least one of whole-exome sequencing (WES) and whole-genome sequencing (WGS); and
(c) determining at least one of a tumor mutational burden and a copy number burden of said subject, based at least in part on processing said set of sequencing reads.

114. A non-transitory computer readable medium comprising machine executable code that, upon execution by one or more computer processors, implements a method comprising:
(a) obtaining or deriving a biological sample from a subject, wherein said subject has cancer, has previously had cancer, or is suspected of having cancer;
(b) assaying cell-free deoxyribonucleic acid (cfDNA) molecules obtained or derived from said biological sample, wherein said assaying comprises sequencing at least a portion of said cfDNA molecules or derivatives thereof to produce a set of sequencing reads, wherein said sequencing comprises at least one of whole-exome sequencing (WES) and whole-genome sequencing (WGS); and
(c) determining at least one of a tumor mutational burden and a copy number burden of said subject, based at least in part on processing said set of sequencing reads.
